(19) United States
(12) Patent Application Publication
(10) Pub. No.: US 2008/0227871 A1
(43) Pub. Date: Sep. 18, 2008

(54) Ampholytic Copolymer, Production Thereof, and Use of the Same

(75) Inventors: Son Nguyen Kim, Hemsbach (DE); Ivette Garcia Castro, Ludwigshafen (DE); Klemens Mathauer, Heidelberg (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/996,904

(22) PCT Filed: Jul. 21, 2006
(86) PCT No.: PCT/EP06/64507

§ 371 Date: Jan. 25, 2008

(30) Foreign Application Priority Data
Jul. 26, 2005 (DE) 10 2005 034 906.4

(51) Int. Cl. A61K 47/34; C08G 77/04

(52) U.S. Cl. 514/772.3; 528/26

(57) ABSTRACT

The following invention relates to copolymers containing silicone groups which comprise, in copolymerized form, at least one monomer with an ionogenic and/or ionic group and at least one crosslinking monomer, to a process for the preparation of such copolymers containing silicone groups by precipitation polymerization, and to the use of these copolymers.

The following invention relates to copolymers containing silicone groups which comprise, in copolymerized form, at least one monomer with an ionogenic and/or ionic group and at least one crosslinking monomer, to a process for the preparation of such copolymers containing silicone groups by precipitation polymerization, and to the use of these copolymers.

Specific requirements are often placed on cosmetic, pharmaceutical and technical compositions with regard to their rheological properties. They can often only be converted to the desired application form using additives, so-called thickeners. Examples of customary low molecular weight thickeners are, for example, the alkali metal and aluminum salts of fatty acids, fatty alcohols or waxes. However, depending on the field of use of the preparation to be thickened, use of the known thickeners is often associated with disadvantages. For example, the thickening effect of the thickeners may not be satisfactory, their use may be undesired or their incorporation into the preparation to be thickened may be hindered or completely impossible, for example due to their incompatibility with the compound to be thickened. The provision of products with a complex profile of properties using the lowest possible fraction or the fewest possible different active substances often presents difficulties. For example, there is a need for polymers for cosmetic and other compositions which have good conditioning properties, i.e. have a positive effect on the sensory properties of the compositions modified therewith, and at the same time allow the rheological properties of the compositions to be adjusted. In addition, esthetic requirements are increasingly being placed on cosmetic and pharmaceutical products by consumers. For example, with such products, a preference for clear, opaque formulations in the form of gels is currently observed. There is therefore a need for cosmetically and pharmaceutically compatible polymers which are suitable for providing a certain profile of properties with regard to the sensory properties and the rheology. These should be able to be converted into powders in particular and nevertheless be capable of being incorporated into a composition to be thickened within a short time and thereby reliably provide the desired rheology properties.

It is known to use polymers to modify the rheological properties. These have the advantage that they generally allow the viscosity to be adjusted depending on their molecular weight. One disadvantage which often arises when using polymers as thickeners for preparing more highly viscous or gel-like preparations is that as the molecular weight of the polymer increases, its incorporation generally becomes more difficult, and that ultimately often only swelling of the polymer is observed instead of the desired solution.

WO 01/85821 describes polyurethanes and their use for modifying rheological properties.

U.S. Pat. No. 3,915,921 describes copolymers which comprise, in copolymerized form, an olefinically unsaturated carboxylic acid, a C₁₀-C₃₀-alkyl (meth)acrylate and, if appropriate, a crosslinking monomer with at least two ethylenically unsaturated double bonds. In neutralized form, they serve as thickeners for diverse applications.

WO 97/21744 describes cross linked anionic copolymers and their use as thickeners and dispersants in aqueous systems.

EP-A-0 982 021 describes the use of (partially) neutralized copolymers of

A) 50 to 99% by weight of monoethylenically unsaturated carboxylic acids and
B) 1 to 50% by weight of at least one comonomer chosen from
a) monoethylenically unsaturated carboxylic esters with saturated C₈-C₃₀-alcohols,
b) N—C₈-C₁₈-alkyl- and N,N-di-C₈-C₁₈-alkylcarboxamides,
c) vinyl esters of aliphatic C₈-C₃₀-carboxylic acids,
d) C₈-C₁₈-alkyl vinyl ethers,
and mixtures thereof
   as thickeners for producing hair-washing compositions.

U.S. Pat. No. 4,395,524 and U.S. Pat. No. 4,432,881 describe copolymers based on monomers containing amide groups which act as thickeners.

DE-A-42 13 971 describes copolymers which comprise, in copolymerized form, at least one olefinically unsaturated monomer containing acid groups, at least one olefinically unsaturated quaternary ammonium compound, if appropriate at least one polyether (meth)acrylate and, if appropriate, at least one crosslinker and their use as thickeners for thickening aqueous systems, which may be cosmetic preparations.

EP-A-893 117 and EP-A-913 143 describe crosslinked cationic copolymers and their use, inter alia, as hair-setting gel formers in cosmetic compositions.

EP-A-1 064 924 describes the use of crosslinked cationic polymers in skin cosmetic and dermatological preparations, inter alia as thickeners.

U.S. Pat. No. 5,015,708 describes a process for the preparation of a terpolymer comprising (i) a vinyllactam, (ii) a monomer containing acid groups and (iii) a hydrophobic monomer, which may, inter alia, be an ethylenically unsaturated silicone compound, by precipitation polymerization, and also the preparation of powders from these polymers.

WO 00/39176 describes a hydrophilic, cationic, ampholytic copolymer which comprises, in copolymerized form, 0.05 to 20 mol % of an anionic monomer with at least one carboxy group, 0 to 45 mol % of a cationic monomer with at least one amino group and, if appropriate, a hydrophobic monomer and/or a crosslinker, where the molar ratio of cationic monomer to anionic monomer is about 2:1 to 16:1. These copolymers containing silicone groups can, inter alia, be used for modifying the rheological properties of body care compositions.

WO 04/058837 describes an ampholytic copolymer which is obtainable by free-radical copolymerization of
a) at least one ethylenically unsaturated compound with at least one anionogenic and/or anionic group,
b) at least one ethylenically unsaturated compound with at least one cationogenic and/or cationic group,
c) at least one unsaturated compound containing amide groups
   and if appropriate further comonomers. The polymerization can take place in the presence of a graft base, which may, inter alia, be a silicone derivative which contains polyalkylene oxide. Also described are polyelectrolyte complexes which comprise such an ampholytic copolymer, and cosmetic or pharmaceutical compositions based on these copolymers which contain silicone groups and polyelectrolyte complexes.

US 2006/0084586 A1 describes rheology-modifying hair-setting resins which comprise a crosslinked copolymer based on vinylamide and carboxylic acid monomers. Polymers which are obtainable by free-radical polymerization in the presence of at least one silicone compound having a polyether group and/or a free-radically polymerizable double bond are not described.

The object of the present invention is to provide novel polymers which are suitable for modifying the rheological properties of cosmetic, pharmaceutical and other compositions. In particular, these polymers should be able to be converted into a solid form, preferably a powder, which can be readily incorporated into the formulations to be thickened. Furthermore, the provided polymers should improve further application properties of the compositions modified therewith, in particular their sensory properties.

Surprisingly, it has now been found that this object is achieved by a copolymer which comprises, in copolymerized form, at least one monomer with at least one ionogenic and/or ionic group, at least one crosslinker and additionally at least one silicone compound.

The invention therefore provides a copolymer A) containing silicone groups, obtainable by free-radical copolymerization of
a) at least one compound with a free-radically polymerizable, α,β-ethylenically unsaturated double bond and at least one ionogenic and/or ionic group per molecule,
b) at least one free-radically polymerizable crosslinking compound which comprises at least two α,β-ethylenically unsaturated double bonds per molecule,
   in the presence of at least one silicone compound c) which comprises a polyether group and/or a free-radically polymerizable olefinically unsaturated double bond.

The copolymers A) containing silicone groups according to the invention can be prepared by conventional polymerization processes, e.g. by solution polymerization or bulk polymerization. Copolymers with particularly advantageous properties, i.e. with generally higher molecular weights and a better ability to form pulverulent formulations than are obtained by conventional polymerization processes are obtained by the preparation in accordance with the method of precipitation polymerization. A preferred embodiment of the invention are therefore copolymers A) containing silicone groups which are obtainable by free-radical copolymerization in accordance with the method of precipitation polymerization in at least one organic solvent. In one specific embodiment, for producing the copolymers according to the invention, use is made of at least two free-radical initiators whose decomposition temperatures and/or whose half-lives at a certain polymerization temperature are different from one another. Here, copolymers with particularly low residual monomer contents can be achieved. This is the case particularly if the initiator which decomposes at a higher temperature is added before completion, preferably before the polymer starts to precipitate.

In the precipitation polymerization, the monomers used are soluble in the reaction medium (monomer, solvent), but the corresponding polymer is not. The polymer which forms becomes insoluble under the polymerization conditions chosen and precipitates out of the reaction mixture. In the process it is possible to obtain ampholytic copolymers A) with molecular weights which are higher than those obtainable by other polymerization processes, e.g. by solution polymerization, which are particularly advantageously suitable as rheology modifiers (specifically thickeners).

For the purposes of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethyl-propyl, n-hexyl, 2-hexyl, 2-methylpentyl, 3-methylpentyl, 4-methylpentyl, 1,2-dimethyl-butyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl groups and C₈-C₃₀-alkenyl groups are straight-chain and branched alkyl groups and alkenyl groups. Preference is given here to predominantly linear alkyl radicals as also occur in natural or synthetic fatty acids and fatty alcohols and in oxo alcohols, which may, if appropriate, additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), arachinyl(ene), behenyl(ene), lignocerinyl(ene), melissinyl(ene), etc.

Cycloalkyl is preferably C₆-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

In the text below, compounds which are derived from acrylic acid and methacrylic acid may sometimes be referred to in short by adding the syllable “(meth)” to the compound derived from acrylic acid.

The copolymers A) according to the invention can advantageously be formulated as gels under normal conditions (20° C.). “Gel-like consistency” is shown by formulations which have a higher viscosity than a liquid and which are self-supporting, i.e. which retain a shape imparted to them without a shape-stabilizing covering. In contrast to solid formulations, gel-like formulations can, however, easily be deformed under the application of shear forces. The viscosity of the gel-like compositions is preferably in a range from greater than 600 to about 60000 mPas, particularly preferably from 6000 to 30000 mPas. The gels are preferably hair gels.

For the purposes of the present invention, water-soluble monomers and polymers are understood as meaning monomers and polymers which dissolve in water at 20° C. in an amount of at least 1 g/l. Water-dispersible monomers and polymers are understood as meaning monomers and polymers which disintegrate into dispersible particles under the application of shear forces, for example by stirring. Hydrophilic monomers are preferably water-soluble or at least water-dispersible. The copolymers A) according to the invention are generally water-soluble.

The copolymers A) according to the invention are particularly advantageously suitable for modifying the rheological properties of compositions which comprise at least one compound which is liquid at 20° C. and 1013 mbar. For the purposes of the present invention, “modification of rheological properties” is understood in the wide sense. Thus, the copolymers A) according to the invention are generally suitable for thickening the consistency of liquid compounds within a wide range. Depending on the basic consistency of the liquid compound(s), flow properties from low viscosity to solid (no longer flowable) are generally achieved depending on the amount of copolymer A) used. “Modification of rheological properties” is therefore understood as meaning, inter alia, the increase in the viscosity of the liquids, the improvement of the thixotropy properties of gels, the solidification of gels and waxes etc.

In a specific embodiment, the copolymers A) containing silicone groups according to the invention have both anionogenic and/or anionic groups and also cationogenic and/or cationic groups. To prepare such copolymers A) containing silicone groups, the oppositely charged/chargeable monomers a) can be used together, i.e. in the form of a monomer pair (“monomer salt”). In this monomer composition, the molar ratio of anionogenic and anionic groups to cationogenic and cationic groups is about 1:1 (i.e. monovalent monomers are essentially used in equimolar amounts). The monomer pairs can be prepared separately prior to being used for the polymerization. However, preference is given to the “in situ” preparation of the monomer pairs through joint use (e.g. joint feed) during the preparation of the copolymers.

### Monomer a)

The copolymers A) containing silicone groups according to the invention comprise, as compound a), at least one compound with a free-radically polymerizable α,β-ethylenically unsaturated double bond and at least one ionogenic and/or ionic group per molecule. The component a) is used preferably in an amount of from 1 to 99% by weight, particularly preferably 5 to 98% by weight, in particular 10 to 97% by weight, based on the total weight of the compounds used for the polymerization (i.e. components a), b), c) and, if present, d) to f)).

In a first preferred embodiment, the copolymers A) comprise, as component a), at least one compound a1) with at least one anionogenic and/or anionic group per molecule in copolymerized form. In a specific embodiment, the monomers a) are exclusively chosen from compounds a1) with anionogenic and/or anionic groups.

Preferably, the component a) comprises at least one compound a1) which is chosen from monoethylenically unsaturated carboxylic acids, sulfonic acids, phosphonic acids and mixtures thereof.

The monomers a1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a1) also include the half-esters of monoethylenically unsaturated dicarboxylic acids having 4 to 10, preferably 4 to 6, carbon atoms, e.g. of maleic acid, such as monomethyl maleate. The monomers a1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers a1) also include the salts of the abovementioned acids, in particular the sodium, potassium and ammonium salts, and the salts with amines. The monomers a1) can be used as they are or as mixtures with one another. The weight fractions given all refer to the acid form.

Preferably, the component a) comprises at least one compound a1) which is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof.

In particular, the component a) comprises at least one compound a1) which is chosen from acrylic acid, methacrylic acid and mixtures thereof.

In a further preferred embodiment, the copolymers A) comprise at least one compound a2) with at least one cationogenic and/or cationic group per molecule in copolymerized form. In a specific embodiment, the component a) consists only of compounds with cationogenic and/or cationic groups.

Preferably, the component a2) comprises at least one compound which is chosen from esters of α,β-ethylenically unsaturated mono- and dicarboxylic acids with amino alcohols which may be mono- or dialkylated on the amine nitrogen, amides of α,β-ethylenically unsaturated mono- and dicarboxylic acids with diamines which have at least one primary or secondary amino group, N,N-diallylamine, N,N-diallyl-N-alkylamines and derivatives thereof, vinyl- and allyl-substituted nitrogen heterocycles, vinyl- and allyl-substituted heteroaromatic compounds and mixtures thereof.

In a particularly preferred embodiment, the component a) comprises, as vinyl-substituted heteroaromatic compound a2), at least one N-vinylimidazole compound. In a specific embodiment, the component a) is chosen from N-vinylimidazole compounds and mixtures which comprise at least one N-vinylimidazole compound.

Preferably, the cationogenic and/or cationic groups of the component a2) are nitrogen-containing groups, such as primary, secondary and tertiary amino groups, and quaternary ammonium groups. The nitrogen-containing groups are preferably tertiary amino groups or quaternary ammonium groups. Charged cationic groups can be produced from the amine nitrogens either by protonation or by quaternization with acids or alkylating agents. These include, for example, carboxylic acids, such as lactic acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate. A protonation or quaternization can generally take place either before or after the polymerization.

Suitable N-vinylimidazole compounds are compounds of the formula

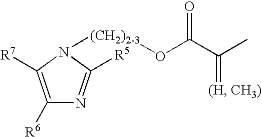

in which R⁵ to R⁷, independently of one another, are hydrogen, C₁-C₄-alkyl or phenyl. Preferably, R⁵ to R⁷ are hydrogen.

Furthermore, the copolymer preferably comprises, as monomer a), at least one N-vinylimidazole compound of the general formula (II)

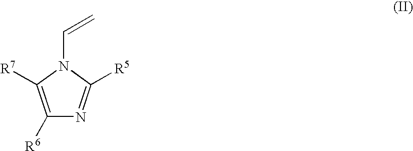

in copolymerized form, in which R⁵ to R⁷, independently of one another, are hydrogen, C₁-C₄-alkyl or phenyl.

Examples of compounds of the general formula (II) are given in Table 1 below:

TABLE 1

| R⁵ | R⁶ | R⁷ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

As monomer a2), preference is given to 1-vinylimidazole (N-vinylimidazole) and mixtures which comprise N-vinylimidazole.

Suitable monomers a2) are also the compounds obtainable by protonation or quaternization of the abovementioned N-vinylimidazole compounds. Examples of such charged monomers a2) are quaternized vinylimidazoles, in particular 3-methyl-1-vinylimidazolium chloride and methosulfate. Suitable acids and alkylating agents are listed below.

Instead of or in addition to the abovementioned N-vinylimidazole compounds, the copolymers A) can comprise at least one other monomer a2) with at least one catiogenic and/or cationic groups in copolymerized form. Preferably, the fraction of these monomers a2) is 0 to 50% by weight, particularly preferably 0 to 30% by weight, very particularly preferably 0.1 to 20% by weight, based on the total weight of the compounds used for the polymerization.

If at least one N-vinylimidazole compound, specifically N-vinylimidazole, is used as the sole monomer a2), then the fraction is preferably 3 to 96% by weight, based on the total weight of the compounds used for the polymerization.

Suitable compounds a2) are the esters of α,β-ethylenically unsaturated mono- and dicarboxylic acids with amino alcohols. Preferred amino alcohols are C₂-C₁₂-amino alcohols which are C₁-C₈-mono- or -dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. As acid component, preference is given to using acrylic acid, methacrylic acid and mixtures thereof.

Preferred monomers a2) are N-tert-butylaminoethyl (meth)acrylate, N N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate. Particular preference is given to N-tert-butylaminoethyl (meth)acrylate and N,N-dimethylaminoethyl (meth)acrylate.

Suitable monomers a2) are also the amides of the abovementioned α,β-ethylenically unsaturated mono- and dicarboxylic acids with diamines which have at least one primary or secondary amino group. Preference is given to diamines which have one tertiary and one primary or secondary amino group.

Preferred monomers a2) are, for example, N-[tert-butylaminoethyl(meth)acrylamide, N-[2-dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide. Particular preference is given to N-[3-(dimethylamino)propyl]acrylamide and N-[3-(dimethylamino)propyl]meth-acrylamide (DMAPMAM).

A specific embodiment relates to copolymers A) which comprise N-[3-dimethyl-amino)propyl]acrylamide and N-[3-(dimethylamino)propyl]methacrylamide and no vinylimidazole compound. In a very specific embodiment, component a2) consists only of N-[3-(dimethylamino)propyl]acrylamide and/or N-[3-(dimethylamino)propyl]meth-acrylamide. The fraction of N-[3-(dimethylamino)propyl]acrylamide and N-[3-(dimethyl-amino)propyl]methacrylamide (in total if both are present) is preferably 2 to 95% by weight, particularly preferably 3 to 60% by weight, based on the total weight of the monomers used for the polymerization.

Suitable monomers a2) are also N,N-diallylamines and N,N-diallyl-N-alkylamines and acid addition salts thereof and quaternization products. Alkyl here is preferably C₁-C₂₄-alkyl. Preference is given to N,N-diallyl-N-methylamine and N,N-diallyl-N,N-dimethylammonium compounds, such as, for example, the chlorides and bromides. Particular preference is given to N,N-diallyl-N-methylamine.

Suitable monomers a2) are also vinyl- and allyl-substituted nitrogen heterocycles different from vinylimidazoles, such as 2- and 4-vinylpyridine, 2- and 4-allylpyridine, and the salts thereof.

### Crosslinker b)

The copolymers A) can, if desired, comprise at least one crosslinker, i.e. a compound with two or more than two ethylenically unsaturated, nonconjugated double bonds in copolymerized form.

Preferably, crosslinkers are used in an amount of from 0.01 to 5% by weight, particularly preferably 0.1 to 4% by weight, based on the total weight of the monomers used for the polymerization.

Suitable crosslinkers b) are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10000. Apart from the homopolymers of ethylene oxide and propylene oxide it is also possible to use block copolymers of ethylene oxide or propylene oxide or copolymers which comprise incorporated ethylene oxide and propylene oxide groups. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted into the corresponding glycidyl ethers by reaction with epichlorohydrin. Preference is given to ethylene glycol di(meth)acrylate and polyethylene glycol di(meth)acrylates.

Further suitable crosslinkers b) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the mono-hydric, unsaturated alcohols with polybasic carboxylic acids, for example malonic acid, tartaric acid, trimellitic acid, phthalic acid, terephthalic acid, citric acid or succinic acid.

Further suitable crosslinkers b) are esters of unsaturated carboxylic acids with the above-described polyhydric alcohols, for example of oleic acid, crotonic acid, cinnamic acid or 10-undecenoic acid.

Suitable crosslinkers b) are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20000.

Also suitable as crosslinkers b) are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids as have been described above.

In addition, triallylamine and triallylmonoalkylammonium salts, e.g. triallylmethylammonium chloride or methyl sulfate, are suitable as crosslinker b).

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers b) are divinyldioxane, tetraallylsilane or tetravinylsilane.

It is of course also possible to use mixtures of the abovementioned compounds b).

As crosslinker b), very particular preference is given to ethylene glycol di(meth)acrylate, polyethylene glycol di(meth)acrylates, pentaerythritol triallyl ether, methylenebisacrylamide, N,N′-divinylethyleneurea, triallylamine and triallylmonoalkylammonium salts.

### Silicone Compound c)

The copolymers A) according to the invention are prepared by polymerization together with at least one silicone compound. The amount of silicone compound c) used is preferably 0.05 to 30% by weight, particularly preferably 0.1 to 20% by weight, in particular 0.5 to 15% by weight, specifically 1 to 10% by weight, based on the total weight of the compounds used for the polymerization.

Suitable silicone compounds c) are either compounds which have a free-radically polymerizable olefinically unsaturated double bond, or compounds which have a polyether group instead of such a double bond. Also suitable are of course compounds which have both at least one free-radically polymerizable double bond and also a polyether group.

In a first embodiment, the free-radical copolymerization to prepare the copolymers A) containing silicone groups takes place in the presence of at least one polyether-containing silicone compound c) which does not comprise α,β-ethylenically unsaturated double bonds. The copolymerization then takes place, in particular, in accordance with the method of precipitation polymerization in at least one organic solvent. The polymerization temperature is then preferably at least 70° C., in particular preferably at least 80° C. Free-radical copolymerization in the presence of such a component c) produces copolymers A) with advantageous properties. This can be attributed, for example, to the effect of the component c) as protective colloid or emulsifier. This can, for example, also result from an at least partial grafting onto the component c) as graft base. However, mechanisms other than grafting are also conceivable. The copolymers A) containing silicone groups according to the invention comprise, quite generally, the process products of the free-radical copolymerization, which is understood as meaning, for example, pure graft polymers, mixtures of graft polymers with ungrafted compounds of the component c), copolymers of the abovementioned monomers, and any mixtures.

Suitable silicone derivatives c) are compounds known under the INCI names dimethicone copolyols or silicone surfactants, such as, for example, the compounds available under the trade names Abil® (from Th. Goldschmidt), Alkasil® (Rhône-Poulenc), Silicone Polyol Copolymer® (Genesee), Belsil® (Wacker), Silwet® (OSI) or Dow Corning (Dow Corning). These include compounds with the CAS numbers 64365-23-7; 68937-54-2; 68938-54-5; 68937-55-3. A suitable commercially available compound is Belsil® DMC 6031.

Particularly suitable compounds c) are those which comprise the following structural elements:

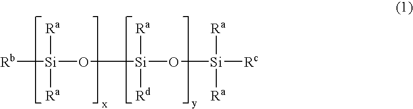

where:
the radicals R^{a} may be identical or different, and are chosen from alkyl, cycloalkyl, cycloalkylalkyl, aryl and arylalkyl, in particular from C₁-C₈-alkyl, C₅-C₈-cycloalkyl, benzyl and phenyl,
R^{b}, R^{c} and R^{d}, independent y of one another, have one of the meanings given above for R^{a} or are —(CH₂)₁₋₆—OH, —(CH₂)₁₋₆—NHR^{e} or a radical of the formula (1.1)

—(CH₂)₁₋₆—O—(CH₂CH₂O)ₐ(CH₂CH(CH₃)O)_{b}—(C═O)_{c}—R^{f} (1.1)

where
in the formula (1.1) the order of the alkylene oxide units is arbitrary,
a and b, independently of one another, are an integer from 0 to 200, where the sum of a and b is >0,
c is 0 or 1,
R^{e} is hydrogen, C₁-C₈-alkyl or C₅-C₈-cycloalkyl,
R^{f} is hydrogen, C₁-C₄₀-alkyl, preferably methyl, or if c=0, may be the anion of an inorganic acid,
   with the proviso that at least one of the radicals R^{b}, R^{c} and R^{d} is a radical of the formula (1.1).

Preferably, x and y are chosen so that the molecular weight of the polysiloxane block is between 300 and 30000.

Preferably, the radical R^{d} is a radical of the formula (1.1).

Preferably, R^{b} and R^{c} are C₁-C₈-alkyl, in particular methyl.

Preferred radicals (1.1) are those in which the sum of a+b is between 5 and 200.

Preferably, the groups R^{a} are chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals, such as benzyl or phenylethyl, and tolyl, xylyl and radicals of the formula (1.1).

Particularly suitable radicals R^{f} are those in which if c=1 R^{f} is any alkyl, cycloalkyl or aryl radical which has between 1 and 40 carbon atoms and which can carry further ionogenic groups, such as NH₂, COOH, SO₃H.

Preferred inorganic radicals R^{f} are, if c=0, phosphate and sulfate.

Particularly preferred silicone derivatives c) are those of the general structure:

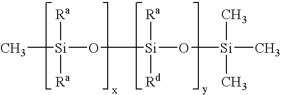

in which R^{d} is a radical of the formula (1.1)

—(CH₂)₁₋₆—O—(CH₂CH₂O)ₐ(CH₂CH(CH₃)O)_{b}—(C═O)_{c}—R^{f} (1.1)

as defined above. Specifically, it is Belsil® DMC 6031 from Wacker.

In a further embodiment, the silicone compound c) is an α,β-ethylenically unsaturated compound which has at least one polysiloxane group.

Suitable ethylenically unsaturated monomers c) with a polysiloxane group are, for example, compounds of the general formula V:

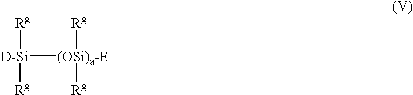

in which
D is an ethylenically unsaturated group which is preferably chosen from vinyl groups, vinyl(C₁-C₄-alkylene) groups, acryloyloxy(C₁-C₄-alkylene) groups and methacryloyloxy(C₁-C₄-alkylene) groups,
R^{g} is in each case, independently of the others, identical or different groups which are chosen from C₁-C₁₀-alkyl, phenyl, benzyl, C₄-C₈-cycloalkyl, and polyalkylene, polyoxyalkylene and polyalkyleneimine groups which can have a terminal alkyl ether, ester or amide function,
E can have the meanings given for R^{g} or those for D and
a is an integer from 1 to 1000, preferably 2 to 250.

Suitable compounds of the formula V are described, for example, in EP-A-0 408 311, which is hereby incorporated in its entirety by reference.

Further suitable silicone compounds c) which have at least one free-radically polymerizable double bond are free-radically polymerizable urethane (meth)acrylates which contain siloxane groups. Of suitability are, for example, the (meth)acrylate-functionalized organopolysiloxane-urethane copolymers described in EP-A-0 274 699 which are obtainable by reacting a polysiloxane functionalized with amino groups with urethane (meth)acrylate oligomers. The disclosure of this document is hereby incorporated by reference.

Preference is given to the compounds described in WO 2004/055088. As compound c) preference is also given to using at least one free-radically polymerizable urethane (meth)acrylate which contains siloxane groups, as described in WO 00/12588. These are urethane (meth)acrylates c) containing siloxane groups which comprise, in incorporated form,
a) at least one compound which comprises at least one active hydrogen atom and at least one free-radically polymerizable, α,β-ethylenically unsaturated double bond per molecule,
b) at least one diisocyanate,
c) at least one compound which comprises two active hydrogen atoms per molecule,
d) at least one compound which comprises at least one active hydrogen atom and at least one siloxane group per molecule,
   and the salts thereof.

For the purposes of the present invention, the expression “urethane (meth)acrylates” comprises, in quite general terms, compounds which have at least one olefinically unsaturated free-radically polymerizable double bond. These also include allylically unsaturated compounds. In addition, the expression “urethane (meth)acrylates” also comprises compounds which have urea groups instead of or in addition to the urethane groups. Urea groups result during the reaction of an isocyanate group with a primary or secondary amino group.

Component c1)

Suitable compounds c1) are, for example, the customary vinyl compounds known to the person skilled in the art which additionally have at least one group which is reactive toward isocyanate groups which is preferably chosen from hydroxyl groups and primary and secondary amino groups. These include, for example, the esters of α,β-ethylenically unsaturated mono- and dicarboxylic acids with at least dihydric alcohols. α,β-Ethylenically unsaturated mono- and/or dicarboxylic acids which may be used are, for example, acrylic acid, methacrylic acid, fumaric acid, maleic acid, crotonic acid, itaconic acid etc. and mixtures thereof. Suitable alcohols are customary diols, triols and polyols, e.g. 1,2-ethanediol, 1,3-propanediol, 1,4-butanediol, 1,5-pentanediol, 1,6-hexanediol, 1,10-decanediol, diethylene glycol, 2,2,4-trimethylpentanediol-1,5,2,2-dimethylpropanediol-1,3,1,4-dimethylolcyclohexane, 1,6-dimethylolcyclohexane, glycerol, trimethylolpropane, erythritol, pentaerythritol, sorbitol etc. The compounds a) are then, for example, hydroxymethyl (meth)acrylate, hydroxyethyl ethacrylate, 2-hydroxyethyl (meth)acrylate, 2-hydroxypropyl (meth)acrylate, 3-hydroxypropyl (meth)acrylate, 3-hydroxybutyl (meth)acrylate, 4-hydroxybutyl (meth)acrylate, 6-hydroxyhexyl (meth)acrylate, 3-hydroxy-2-ethylhexyl (meth)acrylate, and di(meth)acrylic esters of 1,1,1-trimethylolpropane or of glycerol.

Suitable monomers c1) are also the esters and amides of the abovementioned α,β-ethylenically unsaturated mono- and dicarboxylic acids with C₂-C₁₂-amino alcohols which have a primary or secondary amino group. These include aminoalkyl acrylates and aminoalkyl methacrylates and their N-monoalkyl derivatives, which carry, for example, a N—C₁-C₈-monoalkyl radical, such as aminomethyl (meth)acrylate, aminoethyl (meth)acrylate, N-methylaminomethyl (meth)acrylate, N-ethylaminomethyl (meth)acrylate, N-ethylaminoethyl (meth)acrylate, N-(n-propyl)aminomethyl (meth)acrylate, N-isopropylaminomethyl (meth)acrylate and preferably tert-butylaminoethyl acrylate and tert-butylaminoethyl methacrylate. These also include N-(hydroxy-C₁-C₁₂-alkyl)(meth)acrylamides, such as N-hydroxymethyl(meth)acrylamide, N-hydroxyethyl(meth)acrylamide etc.

Suitable monomers c1) are also the amides of the abovementioned α,β-ethylenically unsaturated mono- and dicarboxylic acids with di- and polyamines which have at least two primary or two secondary or one primary and one secondary amino group(s). These include, for example, the corresponding amides of acrylic acid and methacrylic acid, such as aminomethyl(meth)acrylamide, aminoethyl(meth)acrylamide, aminopropyl(meth)acrylamide, amino-n-butyl(meth)acrylamide, methylaminoethyl(meth)acrylamide, ethylaminoethyl(meth)acrylamide, methylaminopropyl(meth)acrylamide, ethylaminopropyl(meth)acrylamide, methylamino-n-butyl(meth)acrylamide etc.

Suitable monomers c1) are also the reaction products of epoxide compounds which have at least one epoxide group with the abovementioned α,β-ethylenically unsaturated mono- and/or dicarboxylic acids and anhydrides thereof. Suitable epoxide compounds are, for example, glycidyl ethers, such as bisphenol A diglycidyl ether, resorcinol diglycidyl ether, 1,3-propanediol diglycidyl ether, 1,4-butanediol diglycidyl ether, 1,5-pentanediol diglycidyl ether, 1,6-hexanediol diglycidyl ether etc.

Component c2)

Component c2) is a customary aliphatic, cycloaliphatic and/or aromatic diisocyanate, such as tetramethylene diisocyanate, hexamethylene diisocyanate, methylenediphenyl diisocyanate, 2,4- and 2,6-tolylene diisocyanate and isomer mixtures thereof, o- and m-xylylene diisocyanate, 1,5-naphthylene diisocyanate, 1,4-cyclohexylene diisocyanate, dicyclohexylmethane diisocyanate and mixtures thereof. Component c2) is preferably hexamethylene diisocyanate, isophorone diisocyanate, o- and m-xylylene diisocyanate, dicyclohexylmethane diisocyanate and mixtures thereof. If desired, up to 3 mol % of the specified compounds can be replaced by triisocyanates.

Component c3)

Suitable compounds of component c3) are, for example, diols, diamines, amino alcohols and mixtures thereof. The molecular weight of these compounds is preferably in a range from about 56 to 280. If desired, up to 3 mol % of the specified compounds can be replaced by triols or triamines.

Suitable diols c3) are, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol.

Suitable amino alcohols c3) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc.

Suitable diamines c3) are, for example, ethylenediamine, propylenediamine, 1,4-diaminobutane, 1,5-diaminopentane and 1,6-diaminohexane.

Preferred compounds of the component c3) are polymers with a number-average molecular weight in the range from about 300 to 5000, preferably about 400 to 4000, in particular 500 to 3000. These include, for example, polyesterdiols, polyetherols, α,ω-diaminopolyethers and mixtures thereof. Preference is given to using polymers containing ether groups.

The polyetherols c3) are preferably polyalkylene glycols, e.g. polyethylene glycols, polypropylene glycols, polytetrahydrofurans etc., block copolymers of ethylene oxide and propylene oxide or block copolymers of ethylene oxide, propylene oxide and butylene oxide which comprise the copolymerized alkylene oxide units in random distribution or in the form of blocks.

Suitable α,ω-diaminopolyethers c3) can be prepared, for example, by amination of polyalkylene oxides with ammonia.

Suitable polytetrahydrofurans c3) can be prepared by cationic polymerization of tetrahydrofuran in the presence of acidic catalysts, such as, for example, sulfuric acid or fluorosulfuric acid. Such preparation processes are known to the person skilled in the art.

Polyesterdiols c3) which can be used preferably have a number-average molecular weight in the range from about 400 to 5000, preferably 500 to 3000, in particular 600 to 2000.

Suitable polyesterdiols are all those which are customarily used for the preparation of polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, and poly(meth)acrylatediols of the formula

HO—C(—R′)(—COOR″)—OH

in which R′ is H or CH₃ and R″ is C₁-C₁₈-alkyl (in particular C₁-C₁₂— or C₁-C₈-alkyl) which have a molar mass of up to about 3000. Diols of this type can be prepared in the usual way and are commercially available (Tegomer® grades MD, BD and OD from Goldschmidt).

Preference is given to polyesterdiols based on aromatic and aliphatic dicarboxylic acids and aliphatic diols, in particular those in which the aromatic dicarboxylic acid constitutes 10 to 95 mol %, in particular 40 to 90 mol % and preferably 50 to 85 mol %, of the total dicarboxylic acid fraction (remainder aliphatic dicarboxylic acids).

Particularly preferred polyesterdiols are the reaction products of phthalic acid/diethylene glycol, isophthalic acid/1,4-butanediol, isophthalic acid/adipic acid/1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane.

The compounds of the component c3) can be used individually or as mixtures.

Component c4)

Preferably, the component c4) is chosen from:
polysiloxanes of the general formula VI.1

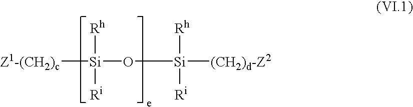

in which
c and d, independently of one another, are 2 to 8,

e is 3 to 100,

R^{h} and Rⁱ, independently of one another, are C₁-C₈-alkyl, benzyl or phenyl,

Z¹ and Z², independently of one another, are OH, NHR^{k} or a radical of the formula VII

—O—(CH₂CH₂O)ᵥ(CH₂CH(CH₃)O)_{w}—H (VII)
where
in the formula VII the order of the alkylene oxide units is arbitrary and v and w, independently of one another, are an integer from 0 to 200, where the sum of v and w is >0,
R^{k} is hydrogen, C₁-C₈-alkyl or C₅-C₈-cycloalkyl;
polysiloxanes of the general formula VI.2

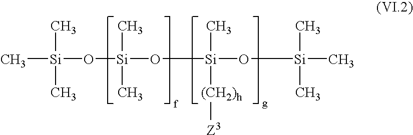

in which
the order of the siloxane units is arbitrary,
f and g, independently of one another, are 0 to 100, where the sum of f and g is at least 2,
h is an integer from 2 to 8,
Z³ is OH, NHR^{k} or a radical of the formula VII,
   where R^{k} is hydrogen, C₁-C₈-alkyl, C₅-C₈-cycloalkyl or a radical of the formula —(CH₂)ᵤ—NH₂, where u is an integer from 1 to 10, preferably 2 to 6,
polysiloxanes with repeat units of the general formula VI.3

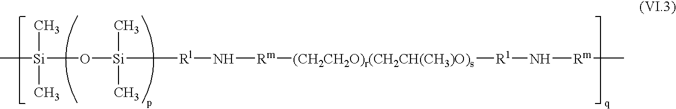

in which
p is an integer from 0 to 100,
q is an integer from 1 to 8,
R^{l} and R^{m}, independently of one another, are C₁-C₈-alkylene,
the order of the alkylene oxide units is arbitrary and
r and s, independently of one another, are an integer from 0 to 200, where the sum of r and s is >0,
polysiloxanes of the general formula VI.4

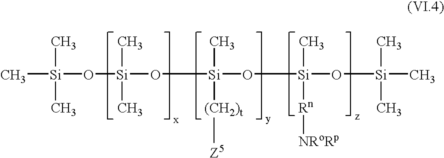

in which
Rⁿ is a C₁-C₈-alkylene radical,
R^{o} and R^{p}, independently of one another, are hydrogen, C₁-C₈-alkyl or C₅-C₈-cycloalkyl,
the order of the siloxane units is arbitrary,
x, y and z, independently of one another, are 0 to 100, where the sum of x, y and z is at least 3,
t is an integer from 2 to 8,
Z⁵ is a radical of the formula VIII

—(OCH₂CH₂)ᵢ(OCH₂CH(CH₃))ⱼ—R^{q} (VIII)
in which
the order of the alkylene oxide units is arbitrary and i and j, independently of one another, are an integer from 0 to 200, where the sum of i and j is >0,
R^{q} is hydrogen or a C₁-C₈-alkyl radical
   and mixtures thereof.

According to a suitable embodiment, the polysiloxanes c4) of the general formula VI.1 have no alkylene oxide radicals of the general formula VII. These polysiloxanes c4) then preferably have a number-average molecular weight in the range from about 300 to 5000, preferably 400 to 3000.

Suitable polysiloxanes c4) which have no alkylene oxide radicals are, for example, the Tegomer® grades from Goldschmidt.

According to a further suitable embodiment, the polysiloxanes c4) are silicone poly(alkylene oxide) copolymers of the formula VI.1, where at least one or two radicals Z¹ and/or Z² are a radical of the general formula VII.

Preferably, in the formula VII, the sum of v and w is chosen so that the molecular weight of the polysiloxanes c4) is then in a range from about 300 to 30000.

Preferably, the total number of alkylene oxide units in the polysiloxanes c4), i.e. the sum of v and w in the formula VII, is then in a range from about 3 to 200, preferably 5 to 180.

According to a further suitable embodiment, the polysiloxanes c4) are silicone poly(alkylene oxide) copolymers of the formula VI.2 which have at least one radical Z³ of the general formula VII.

Preferably, in the formula VII, the sum of v and w is then in turn chosen so that the molecular weight of the polysiloxanes c4) is then in a range from about 300 to 30000. The total number of alkylene oxide units in the polysiloxanes c4), i.e. the sum of v and w in the formula VII, is then preferably likewise in a range from about 3 to 200, preferably 5 to 180.

Suitable silicone poly(alkylene oxide) copolymers c4), which are known under the international generic name dimethicone, are the Tegopren® grades from Goldschmidt, Belsil® 6031 from Wacker and Silvet® L from Witco.

According to a preferred embodiment, the polysiloxanes c4) are silicone poly(alkylene oxide) copolymers of the formula VI.2 which have at least one radical Z³, in which Z³ is NHR^{k} and R³ is hydrogen or a radical of the formula —(CH₂)ᵤ—NH₂. Preferably, u is an integer from 1 to 10, preferably 2 to 6. These include, for example, the MAN and MAR grades from Hüls, and the Finish grades from Wacker, e.g. Finish WT 1270.

Preferably, the polysiloxanes c4) comprise at least one compound of the general formula VI.3. Preferably, in the formula VI.3, R^{l} and R^{m}, independently of one another, are a C₂-C₄-alkylene radical. In particular, R^{l} and R^{m}, independently of one another, are a C₂-C₃-alkylene radical.

Preferably, the molecular weight of the compound of the formula VI.3 is in a range from about 300 to 100000.

Preferably, in the formula VI.3, p is an integer from 1 to 20, such as, for example, 2 to 10.

Preferably, the total number of alkylene oxide units in the compound of the formula VI.3, i.e. the sum of r and s, is in a range from about 3 to 200, preferably 5 to 180.

Preferably, the end groups of the polysiloxanes with repeat units of the general formula VI.3 are chosen from (CH₃)₃SiO, H, C₁-C₈-alkyl and mixtures thereof.

Compounds which contain amino groups and have repeat units of the general formula VI.3 preferably have an amine number in a range from about 2 to 50, in particular 3 to 20.

Suitable alkoxylated siloxane-amines of the formula VI.3 are described, for example, in WO-A-97/32917, which is hereby incorporated in its entirety by reference. Commercially available compounds are, for example, the Silsoft® grades from Witco, e.g. Silsoft® A-843.

Preferably, in the formula VI.4, the radical Rⁿ is a C₂-C₄-alkylene radical.

Preferably, in the formula VI.4 R^{o} and R^{p}, independently of one another, are hydrogen or C₁-C₄-alkyl.

Preferably, the sum of x, y and z is chosen so that the molecular weight of the compound of the formula VI.4 is in a range from about 300 to 100000, preferably 500 to 50000.

Preferably, the total number of the alkylene oxide units in the radical of the formula VIII, i.e. the sum of i and j, is in a range from about 3 to 200, preferably 5 to 80.

Preferably, in the formula VIII, the radical R^{q} is hydrogen or C₁-C₄-alkyl.

A suitable compound of the formula VI.4 is, for example, Silsoft® A-858 from Witco.

Suitable polysiloxanes c4) are also the polydimethylsiloxanes described in EP-A-277 816.

If appropriate, the urethane (meth)acrylates according to the invention additionally comprise at least one incorporated component which is chosen from
c5) compounds which comprise two or more active hydrogen atoms and at least one ionogenic and/or ionic group per molecule,
c6) monohydric alcohols, amines with a primary or secondary amino group, aliphatic, cycloaliphatic or aromatic monoisocyanates and mixtures thereof,
c7) α,β-ethylenically unsaturated compounds which additionally comprise at least one isocyanate group per molecule,
   and mixtures thereof.

### Monomer d)

In a preferred embodiment, the copolymers according to the invention comprise, in copolymerized form, in addition to the abovementioned monomers a) to c), at least one further monomer d) which contains amide groups and is of the general formula I

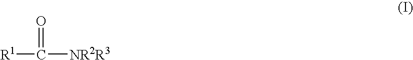

where
one of the radicals R¹ to R³ is a group of the formula CH₂═CR⁴— where R⁴═H or C₁-C₄-alkyl and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8.

Preferably, in addition to the carbonyl carbon atom of the amide group, the compounds of component d) have at most 7 further carbon atoms.

Preferably, the compounds of component d) are chosen from primary amides of α,β-ethylenically unsaturated monocarboxylic acids, N-vinylamides of saturated monocarboxylic acids, N-vinyllactams, N-alkylamides and N,N-dialkylamides of α,β-ethylenically unsaturated monocarboxylic acids and mixtures thereof.

Preferred monomers d) are N-vinyllactams and derivatives thereof, which can, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc.

Particular preference is given to using N-vinylpyrrolidone and N-vinylcaprolactam.

Suitable monomers d) are also acrylamide and methacrylamide.

Suitable N-alkylamides and N,N-dialkylamides of α,β-ethylenically unsaturated monocarboxylic acids which, in addition to the carbonyl carbon atom of the amide group, have at most 7 further carbon atoms are, for example, N-methyl(meth)acrylamide, N-ethyl(meth)acrylamide, N-propyl(meth)acrylamide, N-(n-butyl)(meth)acrylamide, N-tert-butyl(meth)acrylamide, n-pentyl(meth)acrylamide, n-hexyl(meth)acrylamide, n-heptyl(meth)acrylamide, N,N-dimethyl(meth)acrylamide, N,N-diethyl(meth)acrylamide, piperidinyl(meth)acrylamide, morpholinyl(meth)acrylamide and mixtures thereof.

Open-chain N-vinylamide compounds suitable as monomers d) are, for example, N-vinylformamide, N-vinyl-N-methylformamide, N-vinylacetamide, N-vinyl-N-methyl-acetamide, N-vinyl-N-ethylacetamide, N-vinylpropionamide, N-vinyl-N-methyl-propionamide, N-vinylbutyramide and mixtures thereof. Preference is given to using N-vinylformamide.

Suitable monomers d) are also compounds of the formula

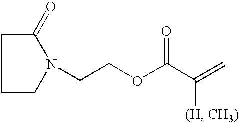

Particular preference is given to using N-vinylpyrrolidone, N-vinylcaprolactam, N-vinylformamide and the compounds of the above formula.

The copolymers A) according to the invention comprise preferably 5 to 95% by weight, particularly preferably 10 to 90% by weight, based on the total weight of the compounds used for the polymerization, of at least one monomer d) in copolymerized form.

### Monomer e)

The copolymers A) according to the invention can additionally comprise at least one hydrophobic monomer e) in copolymerized form. Preferably, the copolymers A) then comprise 0.1 to 30% by weight, particularly preferably 0.2 to 20% by weight, in particular 0.5 to 15% by weight, based on the total weight of the compounds used for the polymerization, of at least one hydrophobic monomer e) in copolymerized form.

Suitable compounds e) are chosen from compounds of the general formulae III a), III b), III c), III d) and III e)

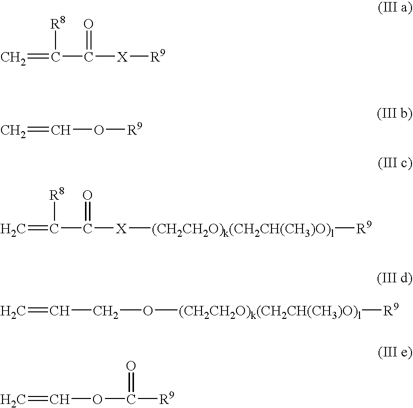

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

Suitable monomers of the formula III a) in which X is O are, for example, n-octyl (meth)acrylate,1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof.

Suitable monomers of the formula III a) in which X is NR¹⁰ are, for example, n-octyl(meth)acrylamide, 1,1,3,3-tetramethylbutyl(meth)acrylamide, ethylhexyl(meth)acrylamide, n-nonyl (meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl meth)acrylamide, stearyl(meth)acrylamide, lauryl(meth)acrylamide, N-methyl-N-(n-octyl)(meth)acrylamide, N,N-di-(n-octyl)(meth)acrylamide and mixtures thereof.

Suitable monomers of the formula III b) are C₈-C₂₂-alkyl vinyl ethers, for example, n-octyl vinyl ether, 1,1,3,3-tetramethylbutyl vinyl ether, ethylhexyl vinyl ether, n-nonyl vinyl ether, n-decyl vinyl ether, n-undecyl vinyl ether, tridecyl vinyl ether, myristyl vinyl ether, pentadecyl vinyl ether, palmityl vinyl ether, heptadecyl vinyl ether, octadecyl vinyl ether, nonadecyl vinyl ether, arrachinyl vinyl ether, behenyl vinyl ether, lignocerenyl vinyl ether, cerotinyl vinyl ether, melissinyl vinyl ether, palmitoleinyl vinyl ether, oleyl vinyl ether, linolyl vinyl ether, linolenyl vinyl ether, stearyl vinyl ether, lauryl vinyl ether and mixtures thereof.

In the formulae III c) and III d), k is preferably an integer from 1 to 500, in particular 3 to 250. l is preferably an integer from 0 to 100.

R⁸ in the formula III c) is preferably hydrogen, methyl, ethyl, n-propyl, isopropyl, n-butyl, sec-butyl, tert-butyl, n-pentyl or n-hexyl, in particular hydrogen, methyl or ethyl.

R⁹ in the formulae III c) and III d) is preferably n-octyl, 1,1,3,3-tetramethylbutyl, ethylhexyl, n-nonyl, n-decyl, n-undecyl, tridecyl, myristyl, pentadecyl, palmityl, hepta-decyl, octadecyl, nonadecyl, arrachinyl, behenyl, lignocerenyl, cerotinyl, melissinyl, palmitoleinyl, oleyl, linolyl, linolenyl, stearyl, lauryl.

Preferably, X in the formula III c) is O or NH.

Suitable polyether acrylates III c) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The polyether acrylates III c) can be used on their own or in mixtures for the preparation of the polymers used according to the invention.

Suitable allyl alcohol alkoxylates III d) are, for example, the etherification products of allyl chloride with corresponding polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The allyl alcohol alkoxylates III d) can be used on their own or in mixtures for the preparation of the polymers used according to the invention.

Suitable monomers III e) are C₈-C₃₀-, preferably C₈-C₂₂-carboxylic acid vinyl esters. These include, for example, n-octyl vinyl ester, 1,1,3,3-tetramethylbutyl vinyl ester, ethylhexyl vinyl ester, n-nonyl vinyl ester, n-decyl vinyl ester, n-undecyl vinyl ester, tridecyl vinyl ester, myristyl vinyl ester, pentadecyl vinyl ester, palmityl vinyl ester, heptadecyl vinyl ester, octadecyl vinyl ester, nonadecyl vinyl ester, arrachinyl vinyl ester, behenyl vinyl ester, lignocerenyl vinyl ester, cerotinyl vinyl ester, melissinyl vinyl ester, palmitoleinyl vinyl ester, oleyl vinyl ester, linolyl vinyl ester, linolenyl vinyl ester, stearyl vinyl ester, lauryl vinyl ester and mixtures thereof.

### Monomer f)

The copolymers A) according to the invention can additionally comprise, in copolymerized form, at least one monomer f) which is chosen from esters of α,β-ethylenically unsaturated mono- and dicarboxylic acids with C₁-C₇-alkanols which are different from component e), polyether acrylates which are different from III c), C₁-C₇-alkyl vinyl ethers, allyl alcohol alkoxylates which are different from III d), and esters of vinyl alcohol with C₁-C₇-monocarboxylic acids.

The fraction of monomers f) is preferably up to 40% by weight, based on the total weight of the compounds used for the polymerization. A suitable use amount of additional monomers f) is in a range from 0.1 to 30% by weight, in particular 1 to 25% by weight, based on the total weight of the compounds used for the polymerization.

Preferably, the compound f) is chosen from compounds of the general formulae III a*) III b*), III c*), III d*) and III e*)

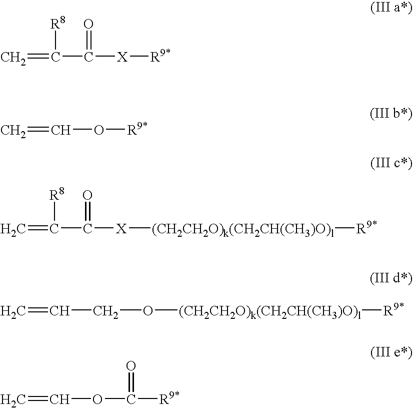

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹* is hydrogen, C₁-C₈-alkyl or C₃-C₈-alkenyl, and
X is O or a group of the formula NR¹⁰, in which R¹⁰ is H, alkyl, alkenyl, cycloalkyl, heterocycloalkyl, aryl or hetaryl.

Suitable esters of α,β-ethylenically unsaturated mono- and dicarboxylic acids with C₁-C₇-alkanols are, for example, methyl (meth)acrylate, methyl ethacrylate, ethyl (meth)acrylate, ethyl ethacrylate, n-butyl (meth)acrylate, tert-butyl (meth)acrylate, tert-butyl ethacrylate, n-pentyl (meth)acrylate, n-hexyl (meth)acrylate, n-heptyl (meth)acrylate, etc. Preferred monomers f) are the esters of α,β-ethylenically unsaturated mono- and dicarboxylic acids with C₁-C₃-alkanols, in particular methyl methacrylate.

In the formulae III c*) and III d*), k is preferably an integer from 1 to 500, in particular 3 to 250. Preferably, l is an integer from 0 to 100.

Preferably, R⁸ in the formula III c*) is hydrogen, methyl, ethyl, n-propyl, isopropyl, n-butyl, sec-butyl, tert-butyl, n-pentyl or n-hexyl, in particular hydrogen, methyl or ethyl.

Preferably, R⁹* in the formulae III c*) and III d*) is hydrogen, methyl, ethyl, n-propyl, isopropyl, n-butyl, sec-butyl, tert-butyl, n-pentyl or n-hexyl, in particular hydrogen, methyl or ethyl.

Preferably, X in the formula III c*) is O or NH.

Suitable polyether acrylates III c*) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with water or a starter alcohol R⁹*—OH. The alkylene oxides can be used individually, alternately one after the other or as a mixture. The polyether acrylates III c*) can be used on their own or in mixtures for the preparation of the polymers used according to the invention.

Suitable allyl alcohol alkoxylates III d*) are, for example, the etherification products of allyl chloride with corresponding polyetherols. Suitable polyetherols can be prepared easily by reacting ethylene oxide, 1,2-propylene oxide and/or epichlorohydrin with water or a starter alcohol R⁹*—OH. The alkylene oxides can be used individually, alternately one after the other or as a mixture. The allyl alcohol alkoxylates III d*) can be used on their own or in mixtures for the preparation of the polymers used according to the invention.

Suitable additional monomers III e*) are also vinyl acetate, vinyl propionate, vinyl butyrate and mixtures thereof.

### Monomer g)

The copolymers A) according to the invention can additionally comprise, in copolymerized form, at least one monomer g) which is different from the components a) to f) and is copolymerizable therewith.

Preferably, the fraction of monomers g) is up to 40% by weight, based on the total weight of the compounds used for the polymerization. A suitable use amount for additional monomers g) is in a range from 0.1 to 25% by weight, in particular 0.5 to 20% by weight, based on the total weight of the compounds used for the polymerization.

Preferably, the component g) is chosen from esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols having a primary or secondary amino group, esters of vinyl alcohol and allyl alcohol with C₁-C₇-mono-carboxylic acids, polyether acrylates different from III c) and III c*), vinylaromatics, vinyl halides, vinylidene halides, C₁-C₈-monoolefins, nonaromatic hydrocarbons with at least two conjugated double bonds and mixtures thereof.

Suitable additional monomers g) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable additional monomers g) are also 2-hydroxyethylacrylamide, 2-hydroxyethylmethacrylamide, 2-hydroxyethylethacrylamide, 2-hydroxy-propylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Suitable polyether acrylates g) are also urethane (meth)acrylates with alkylene oxide groups. Such compounds are described in DE 198 38 851 (component e2)), which is hereby incorporated in its entirety by reference.

Suitable additional monomers g) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The above mentioned additional monomers g) can in each case be used individually or in the form of any desired mixtures.

Particular preference is given to a copolymer A) which comprises, in copolymerized form,
at least 2% by weight of at least one compound a1) with a free-radically polymerizable, α,β-ethylenically unsaturated double bond and at least one anionogenic and/or anionic group per molecule, preferably acrylic acid and/or methacrylic acid,
0.05 to 5% by weight of at least one crosslinker b), preferably ethylene glycol di(meth)acrylate and/or pentaerythritol triallyl ether,
0.05 to 30% by weight of at least one silicone compound c).

Particular preference is also given to a copolymer A) which comprises, in copolymerized form,
at least 2% by weight of at least one compound a1) with a free-radically polymerizable, α,β-ethylenically unsaturated double bond and at least one anionogenic and/or anionic group per molecule, preferably acrylic acid and/or methacrylic acid,
0.05 to 5% by weight of at least one crosslinker b), preferably ethylene glycol di(meth)acrylate and/or pentaerythritol triallyl ether,
0.05 to 30% by weight of at least one silicone compound c),
20 to 95% by weight of vinylpyrrolidone d).

Vinylcaprolactam can also be used instead of or in addition to vinylpyrrolidone.

Particular preference is also given to a copolymer A) which comprises, in copolymerized form,
at least 2% by weight of at least one compound a1) with a free-radically polymerizable, α,β-ethylenically unsaturated double bond and at least one anionogenic and/or anionic group per molecule, preferably acrylic acid and/or methacrylic acid,
0.05 to 5% by weight of at least one crosslinker b), preferably ethylene glycol di(meth)acrylate and/or pentaerythritol triallyl ether,
0.05 to 30% by weight of at least one silicone compound c),
0.1 to 20% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

Particular preference is also given to a copolymer A) which comprises, in copolymerized form,
at least 2% by weight of at least one compound a1) with a free-radically polymerizable, α,β-ethylenically unsaturated double bond and at least one anionogenic and/or anionic group per molecule, preferably acrylic acid and/or methacrylic acid,
0.05 to 5% by weight of at least one crosslinker b), preferably ethylene glycol di(meth)acrylate and/or pentaerythritol triallyl ether,
0.05 to 30% by weight of at least one silicone compound c),
5 to 40% by weight of at least one monomer f) which is preferably chosen from C₁-C₆-(meth)acrylates, in particular methyl (meth)acrylate, ethyl (meth)acrylate, n-butyl (meth)acrylate and mixtures thereof.

The above-described embodiments of the copolymer A) can additionally also comprise at least one compound a2), preferably at least one N-vinylimidazole compound, in copolymerized form. Instead of or in addition to at least one N-vinylimidazole compound, the above described embodiments of copolymer A) can comprise at least one compound a2) different therefrom in copolymerized form. This is preferably chosen from N-[3-(dimethylamino)propyl]acrylamide, N-[3-(dimethylamino)propyl]meth-acrylamide and mixtures thereof.

Particular preference is also given to a copolymer A) which comprises, in copolymerized form,
at least 2% by weight of at least one compound a1) with a free-radically polymerizable, α,β-ethylenically unsaturated double bond and at least one anionogenic and/or anionic group per molecule, preferably acrylic acid and/or methacrylic acid,
0.05 to 5% by weight of at least one crosslinker b), preferably ethylene glycol di(meth)acrylate and/or pentaerythritol triallyl ether,
0.05 to 30% by weight of at least one silicone compound c),
20 to 95% by weight of vinylpyrrolidone d),
0.1 to 20% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
5 to 40% by weight of at least one monomer f) which is preferably chosen from C₁-C₆-(meth)acrylates, in particular methyl (meth)acrylate, ethyl (meth)acrylate, n-butyl (meth)acrylate and mixtures thereof.

Vinylcaprolactam can also be used instead of or in addition to vinylpyrrolidone.

Particularly preferred copolymers A) containing silicone groups are also obtainable by free-radical copolymerization of
at least 2% by weight, based on the total weight of the monomers used for the polymerization, of at least one monomer pair of at least one N-vinylimidazole compound a2) and acrylic acid and/or methacrylic acid a1),
at least 1% by weight, based on the total weight of the monomers used for the polymerization, of at least one additional monomer a1) with an anionogenic or anionic group or at least one additional monomer a2) with a cationogenic or cationic group,
0.05 to 5% by weight, based on the total weight of the monomers used for the polymerization, of at least one free-radically polymerizable crosslinking compound b),
0.05 to 30% by weight, particularly preferably 0.1 to 20% by weight, of at least one silicone compound c),
0 to 95% by weight of at least one monomer d) which contains amide groups,
0 to 40% by weight, based on the total weight of the monomers used for the polymerization, of at least one hydrophobic monomer e),
0 to 40% by weight, based on the total weight of the monomers used for the polymerization, of at least one monomer f).

A specific embodiment of the copolymers A) according to the invention are anionically ampholytic copolymers. These comprise, as component a1), preferably acrylic acid, methacrylic acid or a mixture thereof. N-vinylimidazole, N-[3-dimethylamino)propyl]-acrylamide, N-[3-(dimethylamino)propyl]methacrylamide and mixtures thereof are preferred as component a2).

Preferred anionically ampholytic copolymers A) are obtainable by free-radical copolymerization of
methacrylic acid and/or acrylic acid a1),
at least one compound a2) chosen from N-vinylimidazole, N-[3-(dimethyl-amino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
at least one silicone compound c),
   where the molar ratio of anionogenic/anionic groups to cationogenic/cationic groups is at least 1:1.

Preferred anionically ampholytic copolymers A) are further obtainable by free-radical copolymerization of
methacrylic acid and/or acrylic acid a1),
at least one compound a2) chosen from N-vinylimidazole, N-[3-(dimethyl-amino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
at least one silicone compound c),
3 to 35% by weight, based on the total weight of the monomers used for the polymerization, of at least one monomer f) which is preferably chosen from C₁-C₆-(meth)acrylates,
   where the molar ratio of anionogenic/anionic groups to cationogenic/cationic groups is at least 1.2:1.

Preferred anionically ampholytic copolymers A) are further obtainable by free-radical copolymerization of
methacrylic acid and/or acrylic acid a1),
at least one compound a2) chosen from N-vinylimidazole, N-[3-(dimethyl-amino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
at least one silicone compound c),
0.1 to 10% by weight, based on the total weight of the monomers used for the polymerization, of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
   where the molar ratio of anionogenic/anionic groups to cationogenic/cationic groups is at least 1.4:1.

In the case of the three last-mentioned anionically ampholytic copolymers, up to 60% by weight of component a1), based on the total weight of the monomers a1), can be replaced with at least one monomer d), preferably vinylpyrrolidone and/or vinylcaprolactam.

Preference is further given to anionically ampholytic copolymers A) for whose production at least some of the monomers a1) and a2) are used in the form of a monomer pair.

Particularly preferred anionic copolymers A) containing silicone groups are obtainable by free-radical copolymerization of
at least 2% by weight, based on the total weight of the monomers used for the polymerization, of at least one monomer pair of N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, of at least one silicone compound c),
20 to 95% by weight of vinylpyrrolidone d),
0.1 to 20% by weight of at least one compound e), which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
5 to 40% by weight of at least one monomer f), which is preferably chosen from C₁-C₆-(meth)acrylates, in particular methyl (meth)acrylate, ethyl (meth)acrylate, n-butyl (meth)acrylate and mixtures thereof.

Vinylcaprolactam can also be used instead of or in addition to vinylpyrrolidone. N-[3-(dimethylamino)propyl]acrylamide and/or N-[3-(dimethylamino)propyl]meth-acrylamide can also be used instead of or in addition to vinylimidazole.

Particularly preferred anionic copolymers A) containing silicone groups are also obtainable by free-radical copolymerization of
at least 5% by weight, based on the total weight of the monomers used for the polymerization, of at least one monomer pair of N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, of at least one silicone compound c),
20 to 85% by weight of vinylpyrrolidone d),
5 to 40% by weight of at least one further monomer f), which is preferably chosen from C₁-C₆-(meth)acrylates, in particular methyl (meth)acrylate, ethyl (meth)acrylate, n-butyl (meth)acrylate and mixtures thereof.

Vinylcaprolactam can also be used instead of or in addition to vinylpyrrolidone. N-[3-(dimethylamino)propyl]acrylamide and/or N-[3-(dimethylamino)propyl]meth-acrylamide can also be used instead of or in addition to vinylimidazole.

Particularly preferred anionic copolymers A) containing silicone groups are also obtainable by free-radical copolymerization of
at least 5% by weight, based on the total weight of the monomers used for the polymerization, of at least one monomer pair of N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, of at least one silicone compound c),
20 to 85% by weight of vinylpyrrolidone d),
1 to 20% by weight of at least one further monomer which is chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, and mixtures thereof, in particular from stearyl methacrylate, polyethylene glycol (meth)acrylates terminated with C₁₈-C₂₂-alkyl groups, C₈-C₂₂-carboxylic acid vinyl esters and mixtures thereof.

Vinylcaprolactam can also be used instead of or in addition to vinylpyrrolidone. N-[3-(dimethylamino)propyl]acrylamide and/or N-[3-(dimethylamino)propyl]meth-acrylamide can also be used instead of or in addition to vinylimidazole.

In a specific embodiment, all of the abovementioned anionic copolymers A) containing silicone groups which comprise at least 5% by weight of at least one vinylimidazole compound in copolymerized form are subjected to a partial or complete quaternization. Suitable quaternizing agents are those specified below.

A further specific embodiment of the copolymers A) according to the invention are cationically ampholytic copolymers. These comprise, as component a1), preferably acrylic acid, methacrylic acid or a mixture thereof. N-vinylimidazole and mixtures of N-vinylimidazole with N-[3-(dimethylamino)propyl]acrylamide and/or N-[3-(dimethyl-amino)propyl]methacrylamide are preferred as component a2).

Preferred cationically ampholytic copolymers A) are obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
at least one silicone compound c),
   where the molar ratio of cationogenic/cationic groups to anionogenic/anionic groups is at least 6:1.

Preferred cationically ampholytic copolymers A) are further obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
at least one silicone compound c),
3 to 35% by weight, based on the total weight of the monomers used for the polymerization, of at least one monomer f) which is preferably chosen from C₁-C₆-(meth)acrylates,
   where the molar ratio of cationogenic/cationic groups to anionogenic/anionic groups is at least 6:1.

Preferred cationically ampholytic copolymers A) are further obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
at least one silicone compound c),
0.1 to 10% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
   where the molar ratio of cationogenic/cationic groups to anionogenic/anionic groups is at least 6:1.

In the case of the three last-mentioned cationically ampholytic copolymers, up to 50% by weight of the N-vinylimidazole a2), based on the total weight of component a2), can be replaced by another monomer, preferably N-[3-(dimethylamino)propyl]acrylamide and/or N-[3-(dimethylamino)propyl]methacrylamide.

Preference is also given to cationically ampholytic copolymers A) for whose production at least some of the monomers a1) and a2) are used in the form of a monomer pair.

Particularly preferred cationic copolymers A) containing silicone groups are obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 70% by weight of at least one additional monomer with a cationogenic or cationic group a2), preferably chosen from vinylimidazole compounds, N-[3-(dimethylamino)propyl]acrylamide, N-[3-(dimethylamino)propyl]meth-acrylamide, N,N-dimethylaminoethyl (meth)acrylate, N,N-dimethylaminopropyl (meth)acrylate and mixtures thereof,
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, of at least one silicone compound c),
0 to 95% by weight, preferably 20 to 95% by weight, of vinylpyrrolidone d),
0 to 20% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, and mixtures thereof,
0 to 40% by weight of at least one monomer f) which is preferably chosen from C₁-C₆-(meth)acrylates, in particular methyl (meth)acrylate, ethyl (meth)acrylate, n-butyl (meth)acrylate and mixtures thereof.

Vinylcaprolactam can also be used instead of or in addition to vinylpyrrolidone.

Particularly preferred cationic copolymers A) containing silicone groups are also obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 50% by weight of at least one additional monomer a2) with a cationogenic or cationic group preferably chosen from vinylimidazole compounds, N-[3-(dimethyl-amino)propyl]acrylamide, N-[3-(dimethylamino)propyl]methacrylamide, N,N-dimethylaminoethyl (meth)acrylate, N,N-dimethylaminopropyl (meth)acrylate and mixtures thereof,
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, of at least one silicone compound c),
20 to 95% by weight of vinylpyrrolidone d),
5 to 40% by weight of at least one monomer f) which is preferably chosen from C₁-C₆-(meth)acrylates, in particular methyl (meth)acrylate, ethyl (meth)acrylate, n-butyl acrylate and mixtures thereof.

Vinylcaprolactam can also be used instead of or in addition to vinylpyrrolidone.

Particularly preferred cationic copolymers A) containing silicone groups are also obtainable by free-radical copolymerization of
at least 5% by weight, based on the total weight of the monomers used for the polymerization, of at least one monomer pair of N-vinylimidazole a2) and acrylic acid and/or methacrylic acid a1),
3 to 70% by weight of at least one additional monomer a2) with a cationogenic or cationic group, preferably chosen from vinylimidazole compounds, N-[3-(dimethylamino)propyl]acrylamide, N-[3-(dimethylamino)propyl]meth-acrylamide, N,N-dimethylaminoethyl (meth)acrylate, N,N-dimethylaminopropyl (meth)acrylate and mixtures thereof,
0.1 to 2% by weight of at least one crosslinker c), preferably ethylene glycol di(meth)acrylate and/or pentaerythritol triallyl ether,
0.05 to 30% by weight, particularly preferably 0.1 to 20% by weight, of at least one silicone compound c),
20 to 85% by weight of vinylpyrrolidone,
1 to 20% by weight of at least one further monomer e) which is chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

Vinylcaprolactam can also be used instead of or in addition to vinylpyrrolidone.

In a specific embodiment, all of the abovementioned cationic copolymers A) containing silicone groups are subjected to a partial or complete quaternization. Suitable quaternizing agents are those specified below. Preference is given to anhydrous quaternizing agents. A particularly preferred quaternizing agent is CH₃—Cl.

In a specific embodiment, the free-radical copolymerization of the abovementioned components a) to c) and, if present, d) to g) is carried out in the presence of at least one polyether-containing compound which has no copolymerizable double bond. Here, specific copolymers containing silicone groups and having advantageous properties are obtained. This can be attributed, for example, to the effect of the polyether component as protective colloid or emulsifier. This can, for example, also result from an at least partial grafting onto the polyether component as graft base. However, mechanisms other than grafting are also conceivable. The copolymers containing silicone groups according to the invention comprise, quite generally, the process products of the free-radical copolymerization, which are understood as meaning, for example, pure graft polymers, mixtures of graft polymers with ungrafted compounds of the polyether component, and any desired mixtures.

Preferably, the amount of polyether component used (if present) is 0.1 to 50% by weight, particularly preferably 1 to 25% by weight, based on the total weight of the components used for the polymerization.

Suitable polyether-containing compounds are, for example, water-soluble or water-dispersible nonionic polymers which have alkylene oxide repeat units. The fraction of alkylene oxide repeat units is preferably at least 30% by weight, based on the total weight of the compound. Suitable polyether-containing compounds are, for example, polyalkylene glycols, as are usually also used as nonionic surfactants. Suitable polyalkylene glycols generally have a number-average molecular weight in the range from about 150 to 100000, preferably 300 to 50000, particularly preferably 500 to 40000. Suitable polyalkylene glycols are, for example, polyethylene glycols, polypropylene glycols, polytetrahydrofurans and alkylene oxide copolymers. Suitable alkylene oxides for the preparation of alkylene oxide copolymers are, for example, ethylene oxide, propylene oxide, epichlorohydrin, 1,2- and 2,3-butylene oxide. The alkylene oxide copolymers can comprise the copolymerized alkylene oxide units in random distribution or in the form of blocks. Advantageously, homopolymers of ethylene oxide or copolymers which comprise ethylene oxide are used. Preferably, the fraction of repeat units derived from ethylene oxide is 40 to 99% by weight. Of suitability are, for example, copolymers of ethylene oxide and propylene oxide, copolymers of ethylene oxide and butylene oxide, and copolymers of ethylene oxide, propylene oxide and at least one butylene oxide.

The copolymers A1) are prepared in accordance with customary processes known to the person skilled in the art, e.g. by solution polymerization, precipitation polymerization, suspension polymerization or emulsion polymerization. The W/W polymerization in water with a suitable displacing agent, e.g. a salt, such as NaCl, is also suitable.

Preferred solvents for the solution polymerization are aqueous solvents, such as water and mixtures of water with water-miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of the dihydric alcohols, diethylene glycol, triethylene glycol, polyethylene glycols with number-average molecular weights up to about 3000, glycerol and dioxane. Particular preference is given to the polymerization in water or a water/alcohol mixture, for example in a water/ethanol mixture. The polymerization temperatures in the case of solution polymerization are preferably in a range from about 30 to 120° C., particularly preferably 40 to 100° C.

The copolymers A) containing silicone groups are particularly preferably prepared by precipitation polymerization.

The precipitation polymerization is preferably carried out in a largely anhydrous, aprotic solvent or solvent mixture, preferably in ethyl acetate and/or n-butyl acetate. A largely anhydrous, aprotic solvent or solvent mixture is understood as meaning a solvent or solvent mixture with a water content of at most 5% by weight.

The precipitation polymerization preferably takes place at a temperature in the range from 70 to 140° C., preferably 75 to 100° C., in particular from 80 to 95° C. The resulting polymer particles precipitate out of the reaction solution and can be isolated by customary methods, such as filtration using subatmospheric pressure. For the precipitation polymerization it is possible to use surface-active, polymeric compounds, preferably based on polysiloxanes. In the case of precipitation polymerization, the polymers obtained usually have higher molecular weights than those in the case of solution polymerization.

The polymerization is usually carried out under atmospheric pressure, although it can also proceed under reduced or elevated pressure. A suitable pressure range is between 1 and 5 bar.

To prepare the polymers, the monomers can be polymerized with the help of initiators which form free radicals.

Initiators for the free-radical polymerization which can be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis-(o-toloyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, azobis(2-amidinopropane) dihydrochloride or 2-2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{l}.

In a specific embodiment, for producing the copolymers according to the invention, use is made of at least two free-radical initiators which permit an essentially independent initiation in at least two phases. In this case, copolymers with particularly low residue monomer contents can be achieved.

For the copolymerization, preference is given to using at least two initiators whose decomposition temperatures are different from one another by at least 10° C. Within the scope of the invention, the decomposition temperature is defined as the temperature at which 50% of the molecules decompose into free radicals within 2.5 hours. In the case of this procedure, the copolymerization preferably takes place until completion of the precipitation of the copolymer at a temperature greater than or equal to the lower decomposition temperature and less than the higher decomposition temperature, and, after precipitation, a further reaction takes place at a temperature greater than or equal to the higher decomposition temperature.

Preferably, the method according to the invention comprises a first polymerization phase at a first polymerization temperature and a second polymerization phase at a second polymerization temperature above the first polymerization temperature, where, for the polymerization, at least two initiators are used whose half-lives at the first polymerization temperature differ such that at least one of these initiators decomposes into free radicals during the first polymerization phase and at least one of these initiators does not essentially decompose into free radicals during the first polymerization phase and decomposes into free radicals during the second polymerization phase. Preferably, in the case of this procedure, the second polymerization phase starts essentially after precipitation of the copolymer. “Essentially” after precipitation of the copolymer is understood as meaning that the copolymer is preferably present to at least 80% by weight, preferably at least 90% by weight, in particular at least 95% by weight, based on the total weight of the copolymer, in precipitated form.

The half-life of an initiator can be determined by customary methods known to the person skilled in the art, as described, for example, in the publication “Initiators for high polymers”, Akzo Noble, No. 10737. The half-life of the first polymerization initiator at the first polymerization temperature and of the second polymerization initiator at the second polymerization temperature is preferably in a range from about 1 minute to 3 hours, particularly preferably 5 minutes to 2.5 hours. If desired, shorter half-lives, e.g. from 1 second to 1 minute or longer half-lives than 3 hours can also be used provided it is ensured that the initiator(s) decomposing at the higher temperature essentially decomposes into free radicals during the second polymerization phase.

In addition to the first and second polymerization phase, further polymerization phases can be used at polymerization temperatures different therefrom. Thus, for example, it is possible to carry out a first polymerization phase at a first polymerization temperature which is chosen so that a controlled polymerization (i.e. e.g. avoiding an undesired temperature increase as a result of the heat of reaction, an excessively high reaction rate, etc.) takes place. Subsequently, an after polymerization, for example, can follow at a temperature which is above the first and below the second polymerization temperature and which is chosen so that the initiator(s) decomposing at the higher temperature essentially do not decompose into free radicals. Following completion of this after polymerization, to which the initiator decomposing at the lower temperature and/or another initiator decomposing under the after polymerization conditions can, if desired, be added again, the second polymerization phase can then follow.

Preferably, the initiator system used comprises at least two initiators whose decomposition temperatures differ from one another by at least 15° C.

The initiator decomposing at the lower temperature preferably has a decomposition temperature from 50 to 100° C.

The initiator decomposing at the higher temperature preferably has a decomposition temperature of from 80 to 150° C.

Preferably, the initiator decomposing at the higher temperature is initially introduced at the start of the copolymerization or added before or during the precipitation of the copolymer.

Preferably, the initiator decomposing at the higher temperature is initially introduced at the start of the copolymerization or added before the precipitation of the copolymer.

In the case of a preferred initiator combination, the initiator decomposing at the lower temperature is Trigonox® EHP (bis(2-ethylhexyl)peroxydicarbonate, CAS No. 16111-62-9), and the initiator decomposing at the higher temperature is chosen from tert-butyl peroxypivalate (e.g. Luperox 11 M75 from Atochem), tert-butyl peroctoate, lauroyl peroxide (LPO, CAS No. 105-74-8) or 2,5-dimethyl-2,5-bis(t-butylperoxy)hexane (Trigonox® 101).

A further preferred initiator combination comprises Trigonox® EHP and tert-butyl peroctoate.

A further preferred initiator combination comprises lauroyl peroxide and tert-butyl peroctoate or 2,5-dimethyl-2,5-bis(t-butylperoxy)hexane (Trigonox® 101).

A further preferred initiator combination comprises tert-butyl peroxypivalate (Luperox 11 M75 and tert-butyl peroctoate or 2,5-dimethyl-2,5-bis(t-butylperoxy)hexane (Trigonox® 101).

A further preferred initiator combination comprises tert-butyl peroctoate and 2,5-dimethyl-2,5-bis(t-butylperoxy)hexane (Trigonox® 101).

To achieve the purest polymers possible, the polymers can, for example, be subjected to a washing step with a suitable solvent, e.g. the solvent also used for the polymerization.

The anionogenic groups (acid group s) of the copolymers A) can be partially or completely neutralized with a base. Bases which can be used for the neutralization of the polymers are alkali metal bases such as sodium hydroxide solution, potassium hydroxide solution, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and amines. Suitable amines are, for example, C₁-C₆-alkylamines, preferably n-propylamine and n-butylamine, dialkylamines, preferably diethylpropylamine and dipropyl-methylamine, trialkylamines, preferably triethylamine and triisopropylamine. Preference is given to amino alcohols, e.g. trialkanolamines, such as triethanolamine, alkyl-dialkanolamines, such as methyl- or ethyldiethanolamine and dialkylalkanolamines, such as dimethylethanolamine, and 2-amino-2-methyl-1-propanol. For use in hair-treatment compositions in particular, NaOH, KOH, 2-amino-2-methyl-1-propanol, 2-amino-2-ethylpropane-1,3-diol, diethylaminopropylamine and triisopropanolamine have proven useful for neutralizing the polymers comprising acid groups. The neutralization of the acid groups can also be carried out using mixtures of two or more bases, e.g. mixtures of sodium hydroxide solution and triisopropanolamine. Depending on the intended use, the neutralization can be carried out partially or completely.

Charged cationic groups can be produced from the present cationogenic nitrogen-containing groups either by protonation, e.g. with mono- or polybasic carboxylic acids, such as lactic acid or tartaric acid, or with mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

If the copolymers A) according to the invention are to be both quaternized and also neutralized, then the quaternization is preferably carried out first, followed by the neutralization.

The copolymers A) containing silicone groups according to the invention are advantageously suitable for modifying the rheological properties of aqueous active substance or effect substance compositions. These may, quite generally, be for example cosmetic compositions, pharmaceutical compositions, hygiene products, paints, compositions for the paper industry and the textile industry. In a preferred embodiment, the compositions comprise at least one water-soluble or at least water-dispersible active substance or effect substance. The copolymers A) according to the invention are of course also suitable for modifying the rheological properties of compositions which comprise at least one water-insoluble (hydrophobic) active substance or effect substance.

For the purposes of the present invention, “modifying rheological properties” is understood in the wide sense. The copolymers A) used according to the invention are generally suitable for thickening the consistency of aqueous compositions within a wide range. Depending on the basic consistency of the liquid compositions, flow properties from low viscosity to solid (in the sense of “no longer flowable”) can generally be achieved depending on the amount of the copolymer A) used. “Modifying rheological properties” is therefore understood, inter alia, as meaning the increase in the viscosity of liquids, the improvement in the thixotropy properties of gels, the solidification of gels and waxes etc. The compositions according to the invention are preferably suitable for the formulation of aqueous cosmetic and pharmaceutical products. Preferably, the compositions of the copolymers A) are generally clear. Thus, formulations, in particular cosmetic formulations, can advantageously be colored without impairment by the intrinsic color of the compositions. Furthermore, the compositions can be formulated in the form of opaque to clear gels.

The copolymers A) containing silicone groups according to the invention are specifically suitable as rheology modifiers with properties which can be controlled via the pH. Thus, for example, the abovementioned anionic copolymers A) containing silicone groups are suitable as pH-switchable thickeners for a pH range greater than or equal to 6. The abovementioned cationic copolymers A) containing silicone groups are suitable as pH-switchable thickeners for a pH range of less than or equal to 6.5. Quaternized copolymers A) containing silicone groups which essentially have no protonatable groups are suitable as rheology modifiers in a pH range from about 2 to 10 irrespective of the pH.

The copolymers A) containing silicone groups according to the invention are also specifically suitable as rheology modifiers for salt-containing compositions.

Advantageously, the copolymers A) containing silicone groups according to the invention also act as film-forming and/or conditioning rheology modifiers. They are thus suitable specifically for cosmetic and dermatological compositions, specifically in hair-setting compositions as “setting thickeners” and in hair care compositions as “conditioning thickeners”.

The copolymers A) containing silicone groups are suitable both for the preparation of homogeneous-phase aqueous compositions, and also for the formulation of heterogeneous-phase compositions which additionally comprise at least one water-insoluble (hydrophobic) liquid or solid compound. “Homogeneous-phase compositions” have only a single phase irrespective of the number of their constituents. “Heterogeneous-phase compositions” are disperse systems of two or more immiscible components. These include solid/liquid, liquid/liquid and solid/liquid/liquid compositions, such as dispersions and emulsions, e.g. O/W and W/O formulations which have at least one of the oil or fat components described in more detail below and water as immiscible phases. In principle, the copolymers A) can be used either in the water phase or in the oil phase. In general, heterogeneous-phase liquid/liquid compositions comprise the copolymers A) essentially in the water phase.

The copolymers A) according to the invention are also suitable as solubilizer for essentially water-insoluble compounds. The invention thus further provides the use of a copolymer A) containing silicone groups, as defined above, as solubilizer for the preparation of aqueous formulations of active substances and effect substances which have a solubility in water at 25° C. and 1013 mbar below 10 g/l.

Furthermore, it has been found that the copolymers A) according to the invention are advantageously suitable as protective colloid. The invention thus further provides the use of a copolymer A) containing silicone groups, as defined above, as protective colloid during free-radical aqueous emulsion polymerization.

On the basis of the abovementioned properties, the copolymers A) according to the invention are very generally suitable for the preparation of active substance or effect substance compositions comprising
A) at least one copolymer containing silicone groups, as defined above,
B) at least one active substance or effect substance and
C) if appropriate at least one further active substance different from A) and B), or auxiliary.

Active substances for cosmetics, medicaments, hygiene compositions, textile-treatment compositions etc., i.e. substances which generally develop an effect even at low concentration, e.g. a cosmetic effect on skin and/or hair, a pharmacological effect within an organism, a cleaning and/or disinfectant effect, a modification of a textile, e.g. a crease free finishing, and also effect substances, which impart a certain property to living beings or inanimate substrates, for example color pigments for make-up or emulsion paints, are often formulated and applied in the form of aqueous active substance or effect substance compositions.

The active substance and effect substance compositions comprise the polymer component A) preferably in a fraction of from about 0.001 to 50% by weight, particularly preferably 0.01 to 30% by weight, in particular 0.1 to 20% by weight, based on the total weight of the composition.

The components B) and C) are chosen according to the desired field of use of the composition. Besides components which are typical of the field of use (e.g. certain pharmaceutical active substances), they are chosen from carriers, excipients, emulsifiers, surfactants, preservatives, fragrances, thickeners different from component A), polymers, gel formers, dyes, pigments, photoprotective agents, consistency regulators, antioxidants, antifoams, antistats, resins, solvents, solubility promoters, neutralizing agents, stabilizers, sterilizing agents, propellants, drying agents, opacifiers, etc.

The compositions preferably have a carrier component C) which is chosen from water, hydrophilic components, hydrophobic components and mixtures thereof.

Suitable hydrophilic carriers C) are, for example, mono-, di- or polyhydric alcohols having preferably 1 to 8 carbon atoms, such as ethanol, n-propanol, isopropanol, propylene glycol, glycerol, sorbitol, etc.

Suitable hydrophobic carriers C) are preferably chosen from
i) oils, fats, waxes,
ii) esters of C₆-C₃₀-monocarboxylic acids with mono-, di- or trihydric alcohols which are different from iii),
iii) saturated acyclic and cyclic hydrocarbons,
iv) fatty acids,
v) fatty alcohols,
vi) propellant gases,
   and mixtures thereof.

Suitable silicone oils C) are, for example, linear polydimethylsiloxanes, poly(methylphenylsiloxanes), cyclic siloxanes and mixtures thereof. The number-average molecular weight of the polydimethylsiloxanes and poly(methylphenyl-siloxanes) is preferably in a range from about 1000 to 150000 g/mol. Preferred cyclic siloxanes have 4- to 8-membered rings. Suitable cyclic siloxanes are commercially available, for example, under the name cyclomethicone.

Preferred oil and fat components C) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig fat, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil and fat components.

Suitable cosmetically and pharmaceutically compatible oil and fat components C) are described in Karl-Heinz Schrader, Grundlagen und Rezepturen der Kosmetika [Fundamentals and formulations of cosmetics], 2nd edition, Verlag Hüthig, Heidelberg, pp. 319-355, which is hereby incorporated by reference.

The compositions according to the invention can comprise, as active substance, e.g. as cosmetic and/or pharmaceutical active substance B) (and also if appropriate as auxiliary C)), at least one polymer which differs from the copolymers A) containing silicone groups according to the invention. These include, very generally, anionic, cationic, amphoteric and neutral polymers.

Examples of anionic polymers are homopolymers and copolymers of acrylic acid and methacrylic acid or salts thereof copolymers of acrylic acid and acrylamide and salts thereof; sodium salts of polyhydroxycarboxylic acids, water-soluble or water-dispersible polyesters, polyurethanes, e.g. Luviset PUR® from BASF, and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohol, anionic polysiloxanes, e.g. carboxyfunctional ones, t-butyl acrylate, methacrylic acid (e.g. Luviskol® VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid. Examples of anionic polymers are also vinyl acetate/crotonic acid copolymers, as are sold, for example, under the names Resyn® (National Starch) and Gafset® (GAF), and vinylpyrrolidone/vinyl acrylate copolymers obtainable, for example, under the trade name Luviflex® (BASF). Further suitable polymers are the vinylpyrrolidone/acrylate terpolymer obtainable under the name Luviflex® VBM-35 (BASF) and polyamides containing sodium sulfonate or polyesters containing sodium sulfonate. Also suitable are vinylpyrrolidone/ethyl methacrylate/methacrylic acid copolymers, as are sold by Stepan under the names Stepanhold-Extra and -R1, and the Carboset® grades from BF Goodrich.

Suitable cationic polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviset Clear®, Luviquat Supreme®, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold), cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by reacting polyvinylpyrrolidone with quaternary ammonium compounds), Polymer JR (hydroxyethylcellulose with cationic groups) and cationic polymers based on plants, e.g. guar polymers, such as the Jaguar® grades from Rhodia.

Very particularly suitable polymers are neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers, as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/-methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or water-dispersible polymers, e.g. polyether siloxanes, such as Tegopren® (Goldschmidt) or Belsil® (Wacker).

As already detailed, the copolymers A) are advantageously suitable for stabilizing water-insoluble (or only slightly water-soluble) active substances and effect substances B) in aqueous phase and therefore allow the preparation of aqueous formulations of such active substances and effect substances. They are also suitable for preparing solid formulations of these active substances and effect substances, which can be converted to an aqueous formulation, e.g. commercial form, administration form or active form. This can also take place even after the solid composition has been applied (e.g. in the digestive tract of an organism, etc.).

Within the scope of the present invention, the “solubility improvement” achieved with the polymers A) used according to the invention is therefore understood in the wide sense. It includes, firstly, the stabilization of heterogeneous systems in which the active substance is present as emulsified and/or dispersed phase (disperse phase) in an aqueous medium as continuous phase. It also includes the stabilization of transitional stages to homogeneous solutions, such as colloidal solutions, etc. ranging to molecularly disperse solutions. It also includes a solubility improvement in the sense of a solubilization during which the sparingly water-soluble or water-insoluble substances are converted into clear, at most opalescent aqueous solutions. Finally, it also includes the ability to form so-called “solid solutions”.

A low (poor) solubility means within the scope of this invention a solubility of the active substance or effect substance in water of less than 10 g/l, in particular of less than 1 g/l and specifically of less than 0.1 g/l at 25° C. and 1013 mbar.

The aqueous active substance compositions of water-insoluble active substances or effect substances prepared using the copolymers A) comprise, besides an aqueous medium as continuous phase, at least one active substance and/or effect substance B) which is dispersed or solubilized in the continuous phase and which has a solubility in water at 25° C./1013 mbar of less than 10 g/l, in particular of less than 1 g/l and specifically less than 0.1 g/l, and at least one copolymer A) containing silicone groups.

The active substance is present in the continuous aqueous phase in extremely finely divided form. This can, for example, be attributed to the fact that the active substance forms aggregates in the aqueous phase with the polymers A). These aggregates generally have average particle sizes of less than 1 μm, often of less than 500 nm, in particular of less than 400 nm, specifically of less than 300 nm. Depending on the nature of the polymer and of the active substance or effect substance, and depending on the concentration ratios, the aggregates can also be so small that they are no longer present in the form of detectable discrete particles, but in dissolved form (particle size <10 nm).

The particle sizes given here are weight-average particle sizes, as can be ascertained by dynamic light scattering. Methods for this are known to the person skilled in the art, for example from H. Wiese in D. Distler, Wässrige Polymerdispersionen [Aqueous polymer dispersions], Wiley-VCH 1999, chapter 4.2.1, p. 40ff and literature cited therein, and H. Auweter, D. Horn, J. Colloid Interf. Sci. 105 (1985) 399, D. Lilge, D. Horn, Colloid Polym. Sci. 269 (1991) 704 or H. Wiese, D. Horn, J. Chem. Phys. 94 (1991) 6429.

The copolymers A) can be used advantageously as solubilizers for UV absorbers which are insoluble or sparingly soluble in water.

The term UV absorber comprises, in the context of the present invention, UV-A, UV-B and/or broadband filters.

Advantageous broadband filters, UV-A filter substances or UV-B filter substances are, for example, representatives of the following classes of compounds:

Bisresorcinyltriazine derivatives with the following structure:

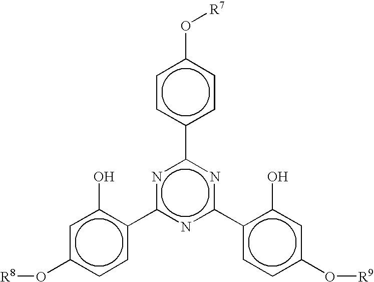

in which R⁷, R⁸ and R⁹ are chosen, independently of one another, from the group of branched and unbranched alkyl groups having 1 to 10 carbon atoms or are a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which can be obtained from CIBA Chemikalien GmbH under the trade name Tinosorb®S.

In addition, other UV filter substances exhibiting the structural unit

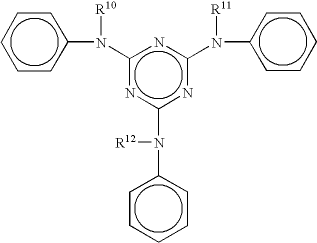

are advantageous UV filter substances for the purposes of the present invention, for example the s-triazine derivatives disclosed in the European Laid-Open Application EP 570 838 A1, the chemical structure of which is represented by the generic formula

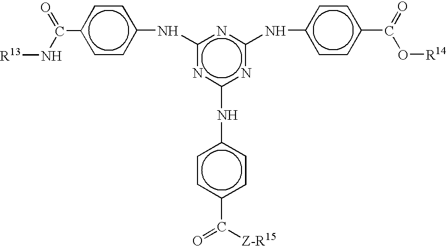

in which
R¹³ is a branched or unbranched C₁-C₁₈-alkyl radical or a C₅-C₁₂-cycloalkyl radical, optionally substituted by one or more C₁-C₄-alkyl groups,
Z is an oxygen atom or an NH group,
R¹⁴ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

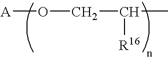

in which
A is a branched or unbranched C₁-C₁₈-alkyl radical, a C₅-C₁₂-cycloalkyl radical or an aryl radical, optionally substituted by one or more C₁-C₄-alkyl groups,
R¹⁶ is a hydrogen atom or a methyl group,
n is a number from 1 to 10,
R¹⁵ is a branched or unbranched C₁-C₁₈-alkyl radical or a C₅-C₁₂-cycloalkyl radical, optionally substituted by one or more C₁-C₄-alkyl groups, if X is the NH group, and is a branched or unbranched C₁-C₁₈-alkyl radical or a C₅-C₁₂-cycloalkyl radical, optionally substituted by one or more C₁-C₄-alkyl groups, or a hydrogen atom, an alkali metal atom, an ammonium group or a group of the formula

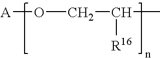

in which
A is a branched or unbranched C₁-C₁₈-alkyl radical, a C₅-C₁₂-cycloalkyl radical or an aryl radical, optionally substituted by one or more C₁-C₄-alkyl groups,
R¹⁶ is a hydrogen atom or a methyl group,
n is a number from 1 to 10,
if X is an oxygen atom.

Furthermore, a particularly preferred UV filter substance for the purposes of the present invention is an asymmetrically substituted s-triazine, the chemical structure of which is represented by the formula

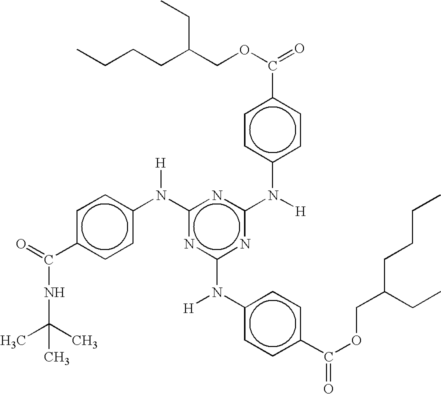

which is also described below as dioctyl butylamido triazone (INCI: Diethylhexyl-butamidotriazone) and is available from Sigma 3V under the trade name UVASORB® HEB.

Also advantageous for the purposes of the present invention is a symmetrically substituted s-triazine, 4,4′,4″-(1,3,5-triazine-2,4,6-triyltriimino)trisbenzoic acid tris(2-ethylhexyl ester), synonym: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150.

In addition, European Laid-Open Application 775 698 discloses bisresorcinyltriazine derivatives which are preferably to be used, the chemical structure of which is represented by the generic formula

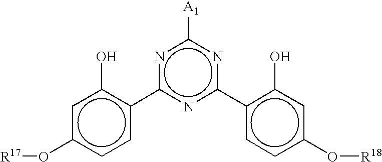

in which R¹⁷ and R¹⁸ are, inter alia, C₃-C₁₈-alkyl or C₂-C₁₈-alkenyl and A₁ is an aromatic radical.

The following compounds are also advantageous for the purposes of the present invention: 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)-phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxy-phenyl)-1,3,5-triazine and 2,4-bis{[4-(1′,1′, 1′,3′,5′,5′,5′-heptamethylsiloxy-2″-methyl-propyloxy)-2-hydroxy]phenyl}-6-(4-methoxyphenyl)-1,3,5-triazine.

Advantageous oil-soluble UV-B and/or broadband filter substances are, e.g.:

3-benzylidenecamphor derivatives, preferably 3-(4-methylbenzylidene)camphor or 3-benzylidenecamphor;
4-aminobenzoic acid derivatives, preferably
4-(dimethylamino)benzoic acid (2-ethylhexyl) ester or
4-(dimethylamino)benzoic acid amyl ester; benzophenone derivatives, preferably 2-hydroxy-4-methoxybenzophenone (available from BASF under the trade name Uvinul® M40), 2-hydroxy-4-methoxy-4′-methylbenzophenone, 2,2′-dihydroxy-4-methoxybenzophenone or 2,2′,4,4′-tetrahydroxybenzophenone (available from BASF under the trade name Uvinul® D 50).

Particularly advantageous U V filter substances for the purposes of the present invention which are liquid at ambient temperature are homomethyl salicylate, 2-ethylhexyl 2-cyano-3,3-diphenylacrylate, 2-ethylhexyl 2-hydroxybenzoate and esters of cinnamic acid, preferably 4-methoxycinnamic acid (2-ethylhexyl) ester and 4-methoxycinnamic acid isopentyl ester.

Homomethyl salicylate (INCI: Homosalate) is characterized by the following structure:

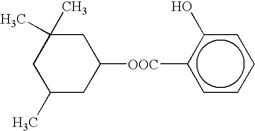

2-Ethylhexyl 2-cyano-3,3-diphenylacrylate (INCI: Octocrylene) is available from BASF under the name Uvinul® N 539T and is characterized by the following structure:

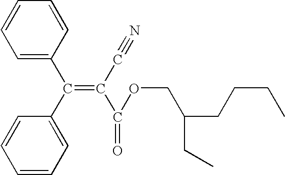

2-Ethylhexyl 2-hydroxybenzoate (2-ethylhexyl salicylate, octyl salicylate, INCI: Ethylhexyl Salicylate) is available, for example, from Haarmann & Reimer under the trade name Neo Heliopan® OS and is characterized by the following structure:

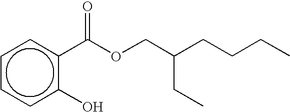

4-Methoxycinnamic acid (2-ethylhexyl) ester (2-ethylhexyl 4-methoxycinnamate, INCI: Ethylhexyl Methoxycinnamate) is, for example, available from BASF under the trade name Uvinul® MC 80 and is characterized by the following structure:

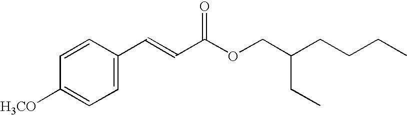

4-Methoxycinnamic acid isopentyl ester (isopentyl 4-methoxycinnamate, INCI: Isoamyl p-Methoxycinnamate) is, for example, available from Haarmann & Reimer under the trade name Neo Heliopan® E 1000 and is characterized by the following structure:

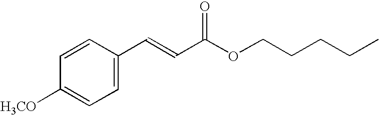

An advantageous dibenzoylmethane derivative for the purposes of the present invention is, in particular, 4-(tert-butyl)-4′-methoxydibenzoylmethane (CAS No. 70356-09-1), which is sold by BASF under the trade name Uvinul® BMBM and by Merck under the trade name Eusolex® 9020 and which is characterized by the following structure:

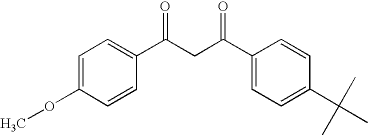

A further advantageous dibenzoylmethane derivative is 4-isopropyl-dibenzoylmethane (CAS No. 63250-25-9), which is sold by Merck under the name Eusolex® 8020. Eusolex 8020 is characterized by the following structure:

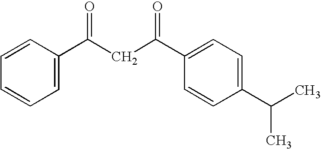

Benzotriazoles are characterized by the following structural formula:

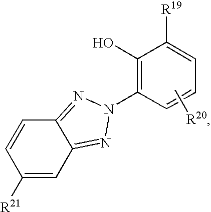

in which
R¹⁹ and R²⁰ are, independently of one another, linear or branched, saturated or unsaturated, substituted (e.g., substituted by a phenyl radical) or unsubstituted alkyl radicals with 1 to 18 carbon atoms.

An advantageous benzotriazole for the purposes of the present invention is furthermore 2-(2H-benzotriazol-2-yl)-4-methyl-6-[2-methyl-3-[1,3,3,3-tetramethyl-1-[(trimethyl-silyl)oxy]disiloxanyl]propyl]phenol (CAS No.: 155633-54-8) with the INCI name Drometrizole Trisiloxane, which is sold by Chimex under the trade name Mexoryl® XL and is characterized by the following structural chemical formula

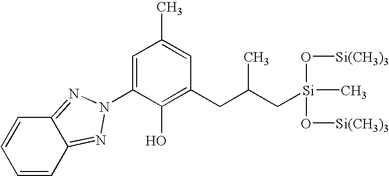

Further advantageous benzotriazoles for the purposes of the present invention are 2,4′-dihydroxy-3-(2H-benzotriazol-2-yl)-5-(1,1,3,3-tetramethylbutyl)-2′-(n-octoxy)-5-benzoyldiphenylmethane, 2,2′-methylenebis[6-(2H-benzotriazol-2-yl)-4-(methyl)-phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di(t-amyl)phenyl)benzo-triazole and 2-(2′-hydroxy-5′-methylphenyl)benzotriazole.

A further UV filter advantageous for the purposes of the present invention is the diphenylbutadiene compound disclosed in EP-A-0 916 335 of the following formula.

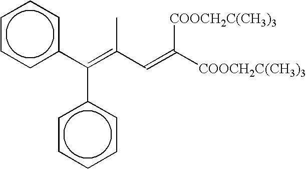

A further UV-A filter which is advantageous for the purposes of the present invention is the 2-(4-ethoxyanilinomethylene)propanedicarboxylic acid diethyl ester disclosed in EP-A-0 895 776 of the following formula.

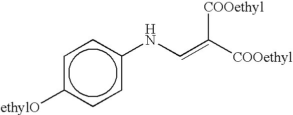

Likewise advantageous for the purposes of the present invention is an amino-substituted hydroxybenzophenone of the following formula:

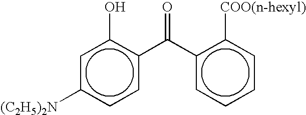

which is sold by BASF Aktiengesellschaft as UV-A filter under the trade name UVINUL® A Plus.

The copolymers A) to be used according to the invention are likewise suitable for the use for modifying the rheological properties, and as solubilizer in pharmaceutical preparations of every type.

The invention therefore further provides a pharmaceutical composition comprising
A) at least one nitrogen atom-containing hyperbranched polymer, as defined above,
B) at least one pharmaceutically acceptable active substance and
C) if appropriate at least one other pharmaceutically acceptable active substance different from B), or auxiliary.

In a specific embodiment, the pharmaceutical compositions comprise at least one pharmaceutically acceptable active substance B) which has a solubility of less than 10 g/l in water at 25° C. and 1013 mbar. For this, the copolymers A) serve as solubilizers for the sparingly soluble active substance(s). The formulation base of the pharmaceutical compositions according to the invention preferably comprises pharmaceutically acceptable auxiliaries. Pharmaceutically acceptable auxiliaries are auxiliaries which are known for use in the field of pharmaceuticals, food technology and related fields, in particular those listed in the relevant pharmacopeias (e.g., DAB, Ph. Eur., BP, NF), and other auxiliaries, the properties of which do not preclude a physiological application.

Suitable auxiliaries can be: lubricants, wetting agents, emulsifying and suspending agents, preservatives, antioxidants, antiirritatives, chelating agents, emulsion stabilizers, film-forming agents, gel formers, odor-masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilants, propellants, drying agents, opacifiers, thickeners, waxes, softeners or white oils. One embodiment relating to this is based on expert knowledge, as described, for example, in Fiedler, H. P., Lexikon der Hilfsstoffe für Pharmazie, Kosmetik und angrenzende Gebiete [Encyclopedia of Auxiliaries for Pharmaceuticals, Cosmetics and Related Fields], 4th edition, Aulendorf: ECV-Editio-Kantor-Verlag, 1996.

In order to prepare pharmaceutical compositions according to the invention, the active substances can be mixed or diluted with a suitable auxiliary (excipient). Excipients can be solid, semiliquid or liquid materials which can act as vehicle, carrier or medium for the active substance. The admixing of additional auxiliaries is carried out, if desired, in a way known to a person skilled in the art. It relates in this connection in particular to aqueous solutions or solubilizates for oral or parenteral application. In addition, the copolymers to be used according to the invention are also suitable for use in oral administration forms, such as tablets, capsules, powders or solutions. In this connection, they can make the sparingly soluble pharmaceutical available with increased bioavailability. In the parenteral application, emulsions, for example fatty emulsions, can also be used in addition to solubilizates. The copolymers A) according to the invention are also suitable for this purpose, in order to process a sparingly soluble pharmaceutical.

Pharmaceutical formulations of the abovementioned kind can be obtained by processing the copolymers A) to be used according to the invention with pharmaceutical active substances using conventional methods and with the use of known and new active substances.

The use according to the invention can additionally comprise pharmaceutical auxiliaries and/or diluents. Cosolvents, astabilizers and preservatives are especially mentioned as auxiliaries.

The pharmaceutical active substances used are substances which are soluble or insoluble or sparingly soluble in water. According to DAB 9 (German Pharmacopeia), the solubility of pharmaceutical active substances is categorized as follows: slightly soluble (soluble in 30 to 100 parts of solvent); sparingly soluble (soluble in 100 to 1000 parts of solvent); virtually insoluble (soluble in more than 10000 parts of solvent). The active substances can in this connection come from any range indicated.

Particular preference is given to those of the abovementioned pharmaceutical compositions relating to formulations which can be applied parenterally.

The content of copolymer A) in the pharmaceutical compositions is, depending on the active substance, in the range from 0.01 to 50% by weight, preferably 0.1 to 40% by weight, particularly preferably 1 to 30% by weight, based on the total weight of the composition.

In principle, all pharmaceutical active substances and prodrugs are suitable for the preparation of the pharmaceutical compositions according to the invention. These include benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, anti parkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, antiinflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgetics, analeptics, aldosterone antagonists and slimming agents. Examples of suitable pharmaceutical active substances are in particular the active substances mentioned in paragraphs 0105 to 0131 of US 2003/0157170.

An additional aspect of the present invention relates to the use of the copolymers A) containing silicone groups as solubilizers in molecularly disperse systems. Solid dispersions, that is homogeneous extremely finely disperse phases of two or more solids, and their special case of “solid solutions” (molecularly disperse systems), and their use in pharmaceutical technology, are generally known (cf. Chiou and Riegelmann, J. Pharm. Sci., 1971, 60, 1281-1300). In addition, the present invention also relates to solid solutions comprising at least one copolymer A) to be used according to the invention.

The preparation of solid solutions can be carried out with the help of melting processes or according to the solution process.

The copolymers according to the invention are suitable as polymeric auxiliary, i.e. solubilizer for the preparation of such solid dispersions or solid solutions.

According to the melting process, for example, an active substance B) and the copolymer A) can be weighed out and mixed in the desired ratio, e.g., in equal parts. A tumbler mixer, for example, is suitable for the mixing. The mixture can subsequently be extruded, e.g. in a twin-screw extruder. The diameter of the cooled product strand thus obtained, consisting of a solid solution of the chosen active substance in the chosen copolymer to be used according to the invention, is dependent on the diameter of the perforation of the perforated plates of the extruder. Cylindrical particles can be obtained by cutting the cooled product strands using a rotating knife, the length of the particles depending on the distance between the perforated plate and the knife. The mean diameter of the cylindrical particles is as a rule approximately 1000 to approximately 3000 μm and the length is as a rule approximately 2000 to approximately 5000 μm. Larger extrudates can be comminuted in a downstream step.

Alternatively, a solid solution can also be prepared in the solution process. For this, the active substance B) and the copolymer A) are usually dissolved in a suitable solvent. Subsequently, the solution is usually poured into a suitable mold and the solvent is removed, for example by drying. The drying conditions are advantageously chosen according to the properties of the active substance (e.g., thermal lability) and solvent (e.g., boiling point).

Taking into consideration the characteristics of the material, the molded article produced or the extrudate, for example, can be comminuted with a suitable mill (e.g., pin mill). The solid solution is advantageously comminuted down to a mean particle size of less than approximately 2000 μm, preferably less than approximately 1000 μm and particularly preferably less than approximately 500 μm.

The bulk material produced can now be processed, with suitable auxiliaries, to give a tableting mixture or to give a capsule feedstock. The tableting is advantageously carried out so that tablets with a hardness of greater than approximately 35 N, preferably greater than approximately 60 N, particularly preferably approximately 80 to approximately 100 N, are obtained.

Like conventional formulations, the formulations thus obtained can, if necessary, be coated with suitable coating materials in order to achieve resistance to gastric juices, delayed release, masking of taste, and the like.

Besides the use in pharmacy, the copolymers A) to be used according to the invention are also suitable in the food sector for modifying the rheological properties and/or as solubilizers for sparingly water-soluble or water-insoluble nutrients, auxiliaries or additives, such as, e.g., fat-soluble vitamins or carotenoids. Mention may be made, as examples, of clear drinks colored with carotenoids. The present invention consequently also provides food preparations comprising at least one of the copolymers A) to be used according to the invention. In the context of the present invention, the food preparations are also to be understood as including food supplements, such as, e.g., preparations comprising food dyes, and dietary foods. Moreover, the specified copolymers A) are also suitable for modifying the rheological properties and/or as solubilizers for feed supplements for animal food.

Furthermore, the copolymers A) containing silicone groups are suitable for the preparation of aqueous preparations of food supplements such as water-insoluble vitamins and provitamins, such as vitamin A, vitamin A acetate, vitamin D, vitamin E, tocopherol derivatives, such as tocopherol acetate, and vitamin K.

Examples of effect substances which can be formulated as aqueous active substance composition according to the invention are dyes: e.g., the dyes disclosed in DE-A 10245209 and the compounds described, according to the Colour Index, as disperse dyes and as solvent dyes, which are also described as dispersion dyes. A list of suitable dispersion dyes is given, for example, in Ullmann's Encyclopedia of Industrial Chemistry, 4th edition, Vol. 10, pp. 155-165 (see also Vol. 7, p. 585ff—Anthraquinone Dyes; Vol. 8, p. 244ff—Azo Dyes; Vol. 9, p. 313ff—Quinophthalone Dyes). This literature reference and the compounds mentioned therein are hereby expressly incorporated by reference. Suitable dispersion dyes and solvent dyes according to the invention comprise the most varied categories of dyes with various chromophores, for example anthraquinone dyes, monoazo and disazo dyes, quinophthalone dyes, methine and azamethine dyes, naphthalimide dyes, naphthoquinone dyes and nitro dyes. Examples of suitable dispersion dyes according to the invention are the dispersion dyes of the following Colour Index list: C. I. Disperse Yellow 1-228, C. I. Disperse Orange 1-148, C. I. Disperse Red 1-349, C. I. Disperse Violet 1-97, C. I. Disperse Blue 1-349, C. I. Disperse Green 1-9, C. I. Disperse Brown 1-21, C. I. Disperse Black 1-36. Examples of suitable solvent dyes according to the invention are the compounds of the following Colour Index list: C. I. Solvent Yellow 2-191, C. I. Solvent Orange 1-113, C. I. Solvent Red 1-248, C. I. Solvent Violet 2-61, C. I. Solvent Blue 2-143, C. I. Solvent Green 1-35, C. I. Solvent Brown 1-63, C. I. Solvent Black 3-50. Suitable dyes according to the invention are furthermore derivatives of naphthalene, of anthracene, of perylene, of terylene or of quarterylene, and diketopyrrolopyrrole dyes, perinone dyes, coumarin dyes, isoindoline and isoindolinone dyes, porphyrin dyes, and phthalocyanine and naphthalocyanine dyes.

In addition to the abovementioned constituents, the active substance and effect substance compositions according to the invention can also comprise conventional surface-active substances and other additives. The surface-active substances include surfactants, dispersing agents and wetting agents. The other additives include in particular thickeners, antifoaming agents, preservatives, antifreeze agents, stabilizers, and the like.

Of use in principle are anionic, cationic, nonionic and amphoteric surfactants, including polymer surfactants and surfactants with heteroatoms in the hydrophobic group.

The anionic surfactants include, for example, carboxylates, in particular alkali metal, alkaline earth metal and ammonium salts of fatty acids, e.g. potassium stearate, which are usually also described as soaps; acyl glutamates; sarcosinates, e.g. sodium lauroyl sarcosinate; taurates; methylcelluloses; alkyl phosphates, in particular mono- and diphosphoric acid alkyl esters; sulfates, in particular alkyl sulfates and alkyl ether sulfates; sulfonates, furthermore alkyl- and alkylarylsulfonates, in particular alkali metal, alkaline earth metal and ammonium salts of arylsulfonic acids and alkyl-substituted arylsulfonic acids, alkylbenzenesulfonic acids, such as, for example, lignin- and phenolsulfonic acid, naphthalene- and dibutylnaphthalenesulfonic acids, or dodecylbenzenesulfonates, alkylnaphthalenesulfonates, alkyl methyl ester sulfonates, condensation products of sulfonated naphthalene and derivatives thereof with formaldehyde, condensation products of naphthalenesulfonic acids, phenol- and/or phenolsulfonic acids with formaldehyde or with formaldehyde and urea, or mono- or dialkylsuccinic acid ester sulfonates; and protein hydrolysates and lignosulfite waste liquors. The abovementioned sulfonic acids are advantageously used in the form of their neutral or, if appropriate, basic salts.

The cationic surfactants include, for example, quaternary ammonium compounds, in particular alkyltrimethylammonium and dialkyldimethylammonium halides and alkyl sulfates, and also pyridine and imidazoline derivatives, in particular alkylpyridinium halides.

The nonionic surfactants include, for example:
fatty alcohol polyoxyethylene esters, for example lauryl alcohol polyoxyethylene ether acetate,
alkyl polyoxyethylene and polyoxypropylene ethers, e.g. of isotridecyl alcohol, and fatty alcohol polyoxyethylene ethers,
alkylaryl alcohol polyoxyethylene ethers, e.g. octylphenol polyoxyethylene ether,
alkoxylated animal and/or plant fats and/or oils, for example corn oil ethoxylates, castor oil ethoxylates or tallow fat ethoxylates,
glycerol esters, such as, for example, glycerol monostearate,
fatty alcohol alkoxylates and oxo alcohol alkoxylates, in particular of the RO—(R₁₈O)ᵣ(R₁₉O)ₛR₂₀ type, with R₁₈ and R₁₉, independently of one another, ═C₂H₄, C₃H₆ or C₄H₈, R₂₀═H or C₁-C₁₂-alkyl, R═C₃-C₃₀-alkyl or C₆-C₃₀-alkenyl, and r and s are, independently of one another, 0 to 50, it not being possible for both to represent 0, such as isotridecyl alcohol and oleyl alcohol polyoxyethylene ether,
alkylphenol alkoxylates, such as, for example, ethoxylated isooctyl-, octyl- or nonylphenol, or tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example, sorbitan fatty acid esters (sorbitan monooleate or sorbitan tristearate), polyoxyethylene sorbitan fatty acid esters, alkylpolyglycosides or N-alkylgluconamides,
alkyl methyl sulfoxides,
alkyldimethylphosphine oxides, such as, for example, tetradecyldimethyl-phosphine oxide.

The amphoteric surfactants include, for example, sulfobetaines, carboxybetaines and alkyldimethylamine oxides, e.g. tetradecyldimethylamine oxide.

Further surfactants which should be mentioned here by way of example are perfluorosurfactants, silicone surfactants, phospholipids such as, for example, lecithin or chemically modified lecithins, or amino acid surfactants, e.g. N-lauroyl glutamate.

Unless otherwise specified, the alkyl chains of the abovementioned surfactants are linear or branched radicals having usually 8 to 20 carbon atoms.

In one embodiment, the aqueous active substance compositions according to the invention comprise no more than 10% by weight, preferably no more than 5% by weight and in particular no more than 3% by weight, e.g. 0.01 to 5% by weight or 0.1 to 3% by weight, of conventional surface-active substances, in each case based on the total amount of active substance and polymer composition. The conventional surface-active substances then preferably make up no more than 5% by weight and in particular no more than 3% by weight, e.g. 0.01 to 5% by weight or 0.1 to 3% by weight, based on the total weight of the composition.

However, depending on the use, it may be advantageous for the active substance compositions according to the invention to be formulated with surface-active substances. The proportion of conventional surface-active substance then frequently lies in the range from 0.5 to 30% by weight, in particular in the range from 1 to 20% by weight, based on the total amount of the active substance and polymer composition, or in the range from 0.2 to 20% by weight and in particular in the range from 0.5 to 15% by weight, based on the total weight of the composition formulated.

Even if one advantage of the compositions according to the invention is their low content of volatile organic substances, it may for some applications be desirable to use the compositions according to the invention with organic solvents, oils and fats, preferably those solvents or oils and fats which are environmentally friendly or biocompatible, e.g. the abovementioned water-miscible solvents or solvents, oils or fats which are immiscible with water or only miscible with water to a very limited extent, e.g.:
paraffin oils, aromatic hydrocarbons and aromatic hydrocarbon mixtures, e.g. xylenes, Solvesso 100, 150 or 200, and the like,
phenols and alkylphenols, e.g. phenol, hydroquinone, nonylphenol, and the like,
ketones with more than 4 carbon atoms, such as cyclohexanone, isophorone, isopherone, acetophenone or acetonaphthone,
alcohols with more than 4 carbon atoms, such as acetylated lanolin alcohol, cetyl alcohol, 1-decanol, 1-heptanol, 1-hexanol, isooctadecanol, isopropyl alcohol, oleyl alcohol or benzyl alcohol,
carboxylic acid esters, e.g. adipic acid dialkyl esters, such as bis(2-ethylhexyl) adipate, phthalic acid dialkyl esters, such as bis(2-ethylhexyl) phthalate, acetic acid alkyl esters (also branched alkyl groups), such as ethyl acetate and ethyl acetoacetate, stearates, such as butyl stearate or glycerol monostearate, citrates, such as tributyl acetylcitrate, in addition cetyl octanoate, methyl oleate, methyl p-hydroxybenzoate, methyl tetradecanoate, propyl p-hydroxybenzoate, methyl benzoate, or lactic acid esters, such as isopropyl lactate, butyl lactate and 2-ethylhexyl lactate,
vegetable oils, such as palm oil, rapeseed oil, ricinus oil and derivatives thereof, such as, e.g., oxidized, coconut oil, cod-liver oil, corn oil, soybean oil, linseed oil, olive oil, peanut oil, safflower oil, sesame seed oil, grapefruit oil, basil oil, apricot oil, ginger oil, geranium oil, orange oil, rosemary oil, macadamia oil, onion oil, mandarin oil, tall oil or sunflower oil,
hydrogenated vegetable oils, such as hydrogenated palm oil, hydrogenated rapeseed oil or hydrogenated soybean oil,
animal oils, such as lard oil or fish oils,
dialkylamides of medium- to long-chain fatty acids, e.g. Hallcomides, and
vegetable oil esters, such as rapeseed oil methyl ester.

The copolymers A) can be used together with conventional thickeners.

Suitable thickeners are compounds which bestow a pseudoplastic flow behavior on the formulation, i.e. high viscosity at rest and low viscosity in the agitated state. Mention may be made, in this connection, for example, of polysaccharides or organic layered minerals, such as Xanthan Gum® (Kelzan® from Kelco), Rhodopol® 23 (Rhône-Poulenc) or Veegum® (R. T. Vanderbilt), or Attaclay® (Engelhardt), with Xanthan Gum® preferably being used.

Silicone emulsions (such as, e.g., Silicone® SRE, from Wacker, or Rhodorsil® from Rhodia), long-chain alcohols, fatty acids, fluoroorganic compounds and their mixtures, for example, come into consideration as antifoam agents suitable for dispersions according to the invention.

Bactericides can be added to stabilize the compositions according to the invention against infection by microorganisms. Suitable bactericides are, for example, Proxel® from ICI or Acticide® RS from Thor Chemie and Kathon® MK from Rohm & Haas.

Suitable antifreeze agents are organic polyols, e.g. ethylene glycol, propylene glycol or glycerol. These are generally used in amounts of no more than 10% by weight, based on the total weight of the active substance composition, in order for the desired content of volatile compounds not to be exceeded. In one embodiment of the invention, the proportion therein of the various volatile organic compounds is preferably no more than 1% by weight, in particular no more than 1000 ppm.

If appropriate, the active substance compositions according to the invention can, to regulate the pH, comprise 1 to 5% by weight of buffer, based on the total amount of the formulation prepared, the amount and the type of the buffer used depending on the chemical properties of the active substance or substances. Examples of buffers are alkali metal salts of weak inorganic or organic acids, such as, e.g., phosphoric acid, boric acid, acetic acid, propionic acid, citric acid, fumaric acid, tartaric acid, oxalic acid and succinic acid.

In a particularly preferred embodiment, the copolymers according to the invention are used as a component in a cosmetic composition. As described previously, they can here serve to modify the rheological properties of a cosmetic composition based on an aqueous medium. They can also serve as solubilizers for cosmetic compositions which comprise at least one cosmetically acceptable active substance or effect substance which has a solubility in water at 25° C. and 1013 mbar of less than 10 g/l.

Independently of this, the copolymers A) according to the invention also have good film-forming properties and as such can also be used as cosmetic active substance.

The invention further provides a cosmetic or pharmaceutical composition comprising

α) at least one copolymer A) containing silicone groups, as defined above, and
β) at least one cosmetically or pharmaceutically acceptable carrier.

Preferably, the component β) is chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols which are different from iii),
v) saturated acyclic and cyclic hydrocarbons,
vi) fatty acids,
vii) fatty alcohols,
viii) propellant gases,
   and mixtures thereof.

Suitable hydrophilic and hydrophobic components β) are those specified above.

Specific suitable cosmetically compatible oil and fat components β) are described in Karl-Heinz Schrader, Grundlagen und Rezepturen der Kosmetika [Fundamentals and formulations of cosmetics], 2nd edition, Verlag Hüthig, Heidelberg, pp. 319-355, which is hereby incorporated by reference.

Preferred hydrophilic carriers β) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygiene or pharmaceutical compositions. On account of their film-forming and thickening properties, the above-described copolymers A) are suitable in particular as additives for hair and skin cosmetics. They are specifically suitable for the formulation of gels.

Preferably, the compositions according to the invention are in the form of a gel, foam, spray, ointment, cream, emulsion, suspension, lotion, milk or paste. If desired, liposomes or microspheres can also be used.

The cosmetically active compositions according to the invention can additionally comprise cosmetically and/or dermatologically active substances and effect substances, and auxiliaries. Of suitability in principle are the abovementioned active substances and effect substances B), and auxiliaries C). In a specific embodiment, the cosmetic compositions according to the invention comprise at least one water-insoluble or only sparingly water-soluble active substance or effect substance.

The cosmetic compositions according to the invention preferably comprise at least one copolymer A) as defined above, at least one carrier β) as defined above and at least one constituent different therefrom which is preferably chosen from cosmetically active substances, emulsifiers, surfactants, preservatives, perfume oils, additional thickeners, hair polymers, hair and skin conditioners, graft polymers, water-soluble or dispersible silicone-containing polymers, photoprotective agents, bleaches, gel formers, care agents, tinting agents, tanning agents, dyes, pigments, consistency regulators, humectants, refatting agents, collagen, protein hydrolysates, lipids, antioxidants, antifoams, antistats, emollients and softeners.

In addition to the copolymers A), suitable conventional thickeners in such formulations are crosslinked polyacrylic acids and derivatives thereof polysaccharides and derivatives thereof such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone.

Suitable cosmetically and/or dermatologically active substances are, for example, skin and hair pigmentation agents, tanning agents, bleaches, keratin-hardening substances, antimicrobial active substances, photofilter active substances, repellent active substances, hyperemic substances, keratolytic and keratoplastic substances, antidandruff active substances, antiphlogistics, keratinizing substances, active substances which act as antioxidants and/or as free-radical scavengers, skin moisturizing or humectant substances, refatting active substances, deodorizing active substances, sebostatic active substances, plant extracts, antierythimatous or antiallergic active substances and mixtures thereof.

Artificially skin-tanning active substances which are suitable for tanning the skin without natural or artificial irradiation with UV rays are, for example, dihydroxyacetone, alloxan and walnut shell extract. Suitable keratin-hardening substances are generally active substances as are also used in antiperspirants, such as, for example, potassium aluminum sulfate, aluminum hydroxychloride, aluminum lactate, etc. Antimicrobial active substances are used in order to destroy microorganisms and/or to inhibit their growth and thus serve both as preservatives and also as deodorizing substance which reduces the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active substances are substances which absorb UV rays in the UV-B and/or UV-A region. Suitable UV filters are those specified above. Also suitable are p-aminobenzoic esters, cinnamic esters, benzophenones, camphor derivatives, and pigments which stop UV rays, such as titanium dioxide, talc and zinc oxide. Suitable repellant active substances are compounds which are able to keep or drive certain animals, in particular insects, away from people. These include, for example, 2-ethyl-1,3-hexanediol, N,N-diethyl-m-toluamide etc. Suitable hyperaemic substances, which stimulate blood flow in the skin, are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenol, etc.

The cosmetic compositions according to the invention can comprise, as cosmetic and/or pharmaceutical active substance (and also if appropriate as auxiliary), at least one cosmetically or pharmaceutically acceptable polymer which differs from the copolymers A) according to the invention. These include, quite generally, anionic, cationic, amphoteric and neutral polymers. The abovementioned polymers are hereby incorporated in their entirety by reference.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, shaving lotions and shaving creams.

According to a further preferred embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eye shadows, lipsticks, kohl pencils, eye liners, blushes, powder and eyebrow pencils.

Furthermore, the copolymers A) can be used in nose strips for pore cleansing, in antiacne compositions, repellents, shaving compositions, hair-removal compositions, intimate care compositions, foot care compositions, and in baby care.

The skin care compositions according to the invention are, in particular, W/O or O/W skin creams, day and night creams, eye creams, face creams, antiwrinkle creams, moisturizing creams, bleach creams, vitamin creams, skin lotions, care lotions and moisturizing lotions.

Skin cosmetic and dermatological compositions based on the above-described copolymers A) exhibit advantageous effects. The polymers can, inter alia, contribute to the moisturization and conditioning of the skin and to the improvement in the feel of the skin. By adding the polymers according to the invention, a considerable improvement in the skin compatibility can be achieved in certain formulations.

Skin cosmetic and dermatological compositions comprise preferably at least one copolymer A) in a fraction of from about 0.001 to 30% by weight, preferably 0.01 to 20% by weight, very particularly preferably 0.1 to 12% by weight, based on the total weight of the composition.

Particularly photoprotective compositions based on the copolymers A) have the property of increasing the residence time of the UV-absorbing ingredients compared to customary auxiliaries such as polyvinylpyrrolidone.

Depending on the field of use, the compositions according to the invention can be applied in a form suitable for skin care, such as, for example, in the form of a cream, foam, gel, stick, mousse, milk, spray (pump spray or propellant-containing spray) or lotion.

Besides the copolymers A) and suitable carriers, the skin cosmetic preparations can also comprise further active substances and auxiliaries customary in skin cosmetics, as described above. These include preferably emulsifiers, preservatives, perfume oils, cosmetic active substances, such as phytantriol, vitamin A, E and C, retinol, bisabolol, panthenol, photoprotective agents, bleaches, tanning agents, collagen, protein hydrolysates, stabilizers, pH regulators, dyes, salts, thickeners, gel formers, consistency regulators, silicones, humectants, refatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with conventional polymers, as described above, if specific properties are to be set.

To set certain properties, such as, for example, improving the feel to the touch, the spreading behavior, the water resistance and/or the binding of active substances and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The cosmetic or dermatological preparations are prepared in accordance with customary processes known to the person skilled in the art.

Preferably, the cosmetic and dermatological compositions are in the form of emulsions, in particular in the form of water-in-oil (W/O) emulsions or oil-in-water (O/W) emulsions. It is, however, also possible to choose other types of formulation, for example hydrodispersions, gels, oils, oleogels, multiple emulsions, for example in the form of W/O/W or O/W/O emulsions, anhydrous ointments and ointment bases, etc.

Emulsions are prepared by known methods. Besides at least one copolymer A), the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The selection of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, which is hereby expressly incorporated by reference.

A suitable emulsion, e.g. for a skin cream etc., generally comprises an aqueous phase which is emulsified by means of a suitable emulsifier system in an oil phase or fat phase. To prepare the aqueous phase, a copolymer A) can be used.

Preferred fat components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start point under atmospheric pressure is at about 250° C. and whose distillation end point is at 410° C., such as, for example, vaseline oil; esters of saturated or unsaturated fatty acids, such as alkyl myristate, e.g. isopropyl myristate, butyl myristate or cetyl myristate, hexadecyl stearate, ethyl or isopropyl palmitate, octanoic acid or decanoic acid triglycerides and cetyl ricinoleate.

The fat phase can also comprise silicone oils which are soluble in other oils, such as dimethylpolysiloxane, methylphenylpolysiloxane and the silicone glycol copolymer, fatty acids and fatty alcohols.

Besides the copolymers A), waxes can also be used, such as, for example, carnauba wax, candililla wax, beeswax, microcrystalline wax, ozokerite wax and Ca, Mg and Al oleates, myristates, linoleates and stearates.

In addition, an emulsion according to the invention can be in the form of an O/W emulsion. Such an emulsion usually comprises an oil phase, emulsifiers which stabilize the oil phase in the water phase, and an aqueous phase, which is usually in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one copolymer A) and usually anionic surfactants as base surfactants and amphoteric and/or nonionic surfactants as cosurfactants. Further suitable active substances and/or auxiliaries are generally chosen from lipids, perfume oils, dyes, organic acids, preservatives and antioxidants, and thickeners/gel formers, skin conditioners and humectants.

These formulations comprise preferably 2 to 50% by weight, preferably 5 to 40% by weight, particularly preferably 8 to 30% by weight of surfactants, based on the total weight of the formulation.

In the washing, shower and bath preparations it is possible to use all of the anionic, neutral, amphoteric or cationic surfactants which are customarily used in body-cleansing compositions.

Suitable anionic surfactants are, for example, alkyl sulfates, alkyl ether sulfates, alkyl-sulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active substances and auxiliaries and water.

According to a further preferred embodiment, the compositions according to the invention are a hair-treatment composition.

Hair-treatment compositions according to the invention comprise preferably at least one copolymer A) in an amount in the range from about 0.1 to 30% by weight, preferably 0.5 to 20% by weight, based on the total weight of the composition.

Preferably, the hair-treatment compositions according to the invention are in the form of a setting foam, hair mousse, hair gel, shampoo, hair spray, hair foam, end fluids, neutralizers for permanent waves or hot oil treatments. Depending on the field of use, the hair cosmetic preparations can be applied in the form of (aerosol) spray, (aerosol) foam, gel, gel spray, cream, lotion or wax. Hair sprays here comprise both aerosol sprays and also pump sprays without propellant gas. Hair foams comprise both aerosol foams and pump foams without propellant gas. Hair sprays and hair foams comprise preferably predominantly or exclusively water-soluble or water-dispersible components. If the compounds used in the hair sprays and hair foams according to the invention are water-dispersible, they can be used in the form of aqueous microdispersions having particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise

a) 0.05 to 20% by weight of at least one copolymer A),
b) 20 to 99.95% by weight of water and/or alcohol,
c) 0 to 50% by weight of at least one propellant gas,
d) 0 to 5% by weight of at least one emulsifier,
e) 0 to 3% by weight of at least one thickener, and
f) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active substances, UV filters, care substances, such as panthenol, collagen, vitamins, protein hydrolysates, alpha- and beta-hydroxycarboxylic acids, stabilizers, pH regulators, dyes, viscosity regulators, gel formers, salts, humectants, refatting agents, complex formers and further customary additives.

Also included here are all styling and conditioner polymers known in cosmetics which can be used in combination with the polymers according to the invention if very specific properties are to be set.

Suitable conventional hair cosmetic polymers are, for example, the abovementioned cationic, anionic, neutral, nonionic or amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds such as amodimethicone (CTFA).

The polymers according to the invention are particularly suitable as setting agents in hair styling preparations, in particular hair foams.

Emulsifiers which may be used are all of the emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 5% by weight of at least one copolymer A),
b) 0 to 5% by weight of at least one cosmetically acceptable water-soluble or water-dispersible hair-setting polymer which is different from A),
c) 80 to 99.85% by weight of water and/or alcohol,
d) 0 to 1% by weight of a gel former different from A),
e) 0 to 20% by weight of further constituents.

Additional gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 allyl ether acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid which are suitable as additional gel formers are commercially available, for example, under the name Carbopol® from BF GOODRICH. Preference is also given to hydrophobically modified crosslinked polyacrylate polymers, such as Carbopol® Ultrez 21 from Noveon. Further examples of anionic polymers which are suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm and Haas.

The copolymers A) according to the invention can be used in cosmetic preparations as conditioners.

Suitable anionic surfactants for the formulation with the copolymers A) are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropyl-betaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the copolymers A). These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacylate, quaternized with diethyl sulfate (Luviquat® PQ 11), copolymers of N-vinylcaprolactam/N-vinylpyrrolidone/-N-vinylimidazolium salts (Luviquat® Hold); cationic cellulose derivatives (Polyquaternium-4 and -10), acrylamide copolymers (Polyquaternium-7). It is also possible to use protein hydrolysates, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and aminofunctional silicone compounds, such as amodimethicone (CTFA). In addition, cationic guar derivatives such as guar hydroxypropyltrimonium chloride (INCI) can be used.

The invention further provides the use of a copolymer A), as defined above, as auxiliary in pharmacy, preferably as or in (a) coating composition(s) for solid medicaments, for modifying rheological properties, as surface-active compound, as or in (an) adhesive(s), and as or in (a) coating composition(s) for the textile, paper, printing and leather industry.

The invention is illustrated in more detail by reference to the following nonlimiting examples.

### Preparation of Silicone-Urethane-Polyallyl

| | | |
|---|---|---|
| Initial charge: | 138.45 g | (0.28 mol) of Pluriol ® A 010R |
| | | (BASF Aktiengesellschaft) |
| | | [allyl alcohol ethoxylate, |
| | | M_{w} = about 500] |
| | 0.50 g | of DABCO |
| Feed 1: | 61.55 g | (0.28 mol) of isophorone diisocyanate |
| Feed 2: | 541.70 g | (0.15 mol) of Pluriol ® ST 4005 |
| | | (BASF Aktiengesellschaft) |
| | | [ethoxylated propoxylated |
| | | polydimethylsiloxane, M_{w} = about 8000] |
| Feed 3: | 184 g | of ethanol |

In a four-necked flask which was fitted with stirrer, dropping funnel, thermometer, reflux condenser and a device for working under nitrogen, Pluriol® A 010R and DABCO were initially introduced under a nitrogen atmosphere and with heating to a temperature of about 60° C. and with stirring. Then, with stirring, over the course of 15 minutes, feed 1 was added dropwise, during which the reaction temperature increased. At a temperature of about 78° C., the reaction mixture was stirred for about 80 minutes. Feed 2 was then metered in over the course of 10 minutes. The reaction mixture was stirred for a further 3 h at about 75° C. (residual NCO content about 0.5%). Feed 3 was then added and the mixture was stirred for a further 30 minutes at 75° C. After cooling to room temperature and adding ethanol, a 75% strength by weight silicone-urethane-polyallyl solution was obtained.

### General Preparation Procedures

### EXAMPLE 5

### (=Variant A1): Polymerization of Silicone-Containing Urethane-Polyallyl/VP/MAA/VI/EGDMA

| | | |
|---|---|---|
| Initial charge: | 412 g | of butyl acetate |
| Feed 1: | 3.6 g | of 75% strength silicone-urethane- |
| | | polyallyl in ethanol |
| | 63.6 g | of vinylpyrrolidone |
| | 67.5 g | of methacrylic acid |
| | 15.0 g | of vinylimidazole |
| | 1.2 g | of ethylene glycol dimethacrylate |
| Feed 2: | 38.2 g | of butyl acetate |
| | 0.15 g | of tert-butyl peroctoate |
| Feed 3: | 95.6 g | of butyl acetate |
| | 0.39 g | of tert-butyl peroctoate |
| Feed 4: | 70 g | of methyl chloride |

In an apparatus which was fitted with stirrer, reflux condenser, internal thermometer and four feed devices, feed 1 and feed 2 were added at 85 to 88° C. over the course of two hours. The resulting reaction mixture was stirred for a further 2 hours at about 88° C. Feed 3 was then metered in over 30 minutes. The reaction mixture was after-polymerized for a further 3 hours at 90° C. The product which precipitated out as white powder was quaternized with methyl chloride (feed 4) at 90° C. over about 1 h. The powder obtained was then filtered off with suction over a suction filter, washed twice with acetone and dried at 40° C. under reduced pressure.

The polymers 11, 15, 26 and 33 given in Table 1 were prepared analogously.

### EXAMPLE 6

### (=Variant A2): Polymerization of VP/MAA/VI/EGDMA in the Presence of an Ethoxylated Dimethylsiloxane

| | | |
|---|---|---|
| Initial charge: | 412 g | of butyl acetate |
| | 1.5 g | of Belsil ® DMC 6031 |
| Feed 1: | 64.2 g | of vinylpyrrolidone |
| | 67.5 g | of methacrylic acid |
| | 15.0 g | of vinylimidazole |
| | 1.8 g | of ethylene glycol dimethacrylate |
| Feed 2: | 38.2 g | of butyl acetate |
| | 0.15 g | of tert-butyl peroctoate |
| Feed 3: | 95.6 g | of butyl acetate |
| | 0.39 g | of tert-butyl peroctoate |
| Feed 4: | 23.4 g | of triethanolamine (about 20%, |
| | | based on methacrylic acid) |

In a stirred apparatus which was fitted with reflux condenser, internal thermometer and four feed devices, 1.5 g of Belsil® DMC 6031 (Wacker) in 412 g of butyl acetate were initially introduced. At 85 to 88° C., feed 1 and feed 2 were metered in over the course of two hours. The resulting reaction mixture was stirred at about 88° C. for 2 h. Feed 3 was then metered in over the course of 30 minutes. The reaction mixture was after-polymerized for a further 3 hours at 90° C. After cooling to about 40° C., the product which precipitated out as a white powder was partially neutralized with triethanolamine (feed 4) over 1 h at 40° C. or quaternized with methyl chloride (feed 4) over about 1 h at 90° C. The powder was filtered off with suction via a suction filter, washed twice with acetone and dried under reduced pressure at 40° C.
The polymers 1-4, 7-10, 12-14, 16-25, 27-32 and 34-50 given in Table 1 were prepared analogously.

The polymers given in Table 1 below can also be produced particularly advantageously by the following production variants. Here, polymers with low residual monomer contents are usually obtained.

| Polymer | Variant |
|---|---|
| 1 | D |
| 2 | A2 |
| 3 | B |
| 4 | B |
| 5 | A1 |
| 6 | A2 |
| 7 | B |
| 8 | B |
| 9 | B |
| 10 | B |
| 11 | A1 |
| 12 | B |
| 13 | C |
| 14 | C |
| 15 | A1 |
| 16 | B |
| 17 | B |
| 18 | B |
| 19 | B |
| 20 | C |
| 21 | B |
| 22 | B |
| 23 | B |
| 24 | B |
| 25 | B |
| 26 | B |
| 27 | B |
| 28 | B |
| 29 | B |
| 30 | B |
| 31 | B |
| 32 | B |
| 33 | B |
| 34 | B |
| 35 | B |
| 36 | B |
| 37 | B |
| 38 | B |
| 39 | B |
| 40 | B |
| 41 | B |
| 42 | B |
| 43 | B |
| 44 | B |
| 45 | B |
| 46 | B |
| 47 | B |
| 48 | B |
| 49 | B |
| 50 | B |

TABLE I

| | (EO) | | | | | | | | |
|---|---|---|---|---|---|---|---|---|---|
| Ex. | silicone^{#} | SI-UPA^{#} | VP^{#} | VCap^{#} | MAA^{#} | AA^{#} | VI^{#} | DMAPMAM^{#} | SMA^{#} |
| 1 | 1.0 | — | — | — | — | 94 | — | — | 4 |
| 2 | 1.0 | — | — | — | 20 | 58.5 | — | — | — |
| 3 | 1.0 | — | 45 | — | — | 25 | — | — | 10 |
| 4 | 1.0 | — | 43 | — | 45 | — | — | — | — |
| 5 | — | 1.8 | 42.4 | — | 45 | — | 10 | — | — |
| 6 | 1.0 | — | 42.8 | — | 45 | — | 10 | — | — |
| 7 | 3.0 | — | 42.8 | — | 40 | — | 8 | — | — |
| 8 | 5.0 | — | 42.8 | — | 40 | — | 6 | — | — |
| 9 | 1.0 | — | 44 | — | 45 | — | 9 | — | — |
| 10 | 1.8 | — | 44 | — | 42 | — | 9 | — | 2 |
| 11 | — | 2.0 | 44 | — | 42 | — | 9 | — | 2 |
| 12 | 2.0 | — | 43 | — | 30 | 15 | — | 10 | — |
| 13 | 2.0 | — | 43 | — | 30 | 15 | 10 | — | — |
| 14 | 2.0 | — | 55 | — | 20 | 10 | 5 | — | 8 |
| 15 | — | 2.2 | 60 | — | 25 | — | 5 | — | — |
| 16 | 0.5 | — | 33.6 | — | 44.8 | — | 9.95 | — | — |
| 17 | 1.0 | — | 78.6 | — | 7 | — | 10 | — | 3 |
| 18 | 3.5 | — | 73 | — | 5 | — | 8 | — | — |
| 19 | 5 | — | 83 | — | 3 | — | 8 | — | 0.5 |
| 20 | 2 | — | 60 | — | 10 | — | — | 22.5 | — |
| 21 | 1 | — | 70 | — | 4 | — | 22 | — | 2.5 |
| 22 | 2.0 | — | 42.9 | — | 44.1 | | 9.8 | — | — |
| 23 | 1.0 | — | 33.5 | 9.9 | 44.5 | — | 9.9 | — | — |
| 24 | 1.0 | — | 43.2 | — | 44.5 | — | 9.9 | — | — |
| 25 | 0.5 | — | 43.4 | — | 44.8 | — | 9.9 | — | — |
| 26 | — | 1.7 | 70 | — | 4 | — | 22 | — | 2 |
| 27 | 1 | — | 70 | — | 3.5 | — | 15 | — | — |
| 28 | 1.5 | — | 50 | 23 | 5 | — | 20 | — | — |
| 29 | 4.5 | — | 25 | 25 | 5 | — | 40 | — | — |
| 30 | 5 | — | — | — | 10 | — | 75 | — | — |
| 31 | 1.5 | — | — | — | 5 | — | 78 | — | — |
| 32 | 0.5 | — | 38.6 | — | 44.8 | — | 5.0 | — | — |
| 33 | — | 1.7 | — | — | 5 | — | 83 | — | — |
| 34 | 0.5 | — | 38.6 | — | 44.8 | — | 14.9 | — | — |
| 35 | 0.5 | — | 43.8 | — | 39.8 | 9.9 | 5 | — | — |
| 36 | 2 | — | 42.9 | — | 44.1 | — | 9.8 | — | — |
| 37 | 1.4 | — | — | — | — | — | 83 | — | 5 |
| 38 | 4.5 | — | — | — | — | — | 85 | — | — |
| 39 | 1.5 | — | — | — | — | — | 83 | — | — |
| 40 | 1.5 | — | 30 | — | — | — | 53 | — | — |
| 41 | 2 | — | 33.1 | — | 44.1 | — | 9.8 | — | — |
| 42 | 1.5 | — | 50 | — | — | — | 33 | — | — |
| 43 | 0.5 | — | 59 | — | — | — | 25 | — | — |
| 44 | 1 | — | 37.8 | — | 45 | — | 5 | — | — |
| 45 | 1 | — | 25.8 | — | 45 | — | 7 | — | — |
| 46 | 1 | — | 12.8 | — | 45 | — | 10 | — | — |
| 47 | 1 | — | — | — | 45 | 7.8 | 10 | — | — |
| 48 | 1 | — | 37.8 | 10 | 40 | — | 5 | — | — |
| 49 | 1 | — | 32.8 | — | 45 | — | 10 | — | — |
| 50 | 1 | — | 27.8 | — | 45 | — | 15 | — | — |

| | | | | | | | | Quat. | Neut. |
|---|---|---|---|---|---|---|---|---|---|
| | | C₁₈- | | | | | | with | with |
| | | PEG- | | | | PEG | Lutensol | CH₃—Cl | TEA |
| Ex. | MMA^{#} | MA^{#} | BMA | EGDMA^{#} | PETAE^{#} | 9000^{#} | AT25^{#} | [%] | DN [%] |
| 1 | — | — | | 1.0 | — | — | — | — | 20 |
| 2 | 15 | 5 | | — | 0.5 | — | — | — | 20 |
| 3 | — | — | | — | 0.5 | 18.5 | — | — | 20 |
| 4 | 7.5 | 2.5 | | 1.0 | — | — | — | — | 20 |
| 5 | — | — | | 0.8 | — | — | — | 75 | — |
| 6 | — | — | | 1.2 | — | — | — | — | 20 |
| 7 | — | — | | 1.2 | — | — | 5 | — | 20 |
| 8 | — | — | | 1.2 | — | — | 5 | — | 20 |
| 9 | — | — | | 1.0 | — | — | — | 75 | — |
| 10 | — | — | | 1.2 | — | — | — | — | 20 |
| 11 | — | — | | 1.0 | — | — | — | — | 20 |
| 12 | — | — | | 1.2 | — | — | — | 75 | — |
| 13 | — | — | | 1.2 | — | — | — | 75 | — |
| 14 | — | — | | 1.2 | — | — | — | — | 20 |
| 15 | 6 | 2 | | 0.8 | — | — | — | — | 20 |
| 16 | — | — | 9.95 | 1.2 | — | — | — | ≧70 | — |
| 17 | — | — | | — | 0.4 | — | — | 75 | — |
| 18 | 7.5 | 2.5 | | — | 0.5 | — | — | 75 | — |
| 19 | — | — | | — | 0.5 | — | — | 75 | — |
| 20 | 3.75 | 1.25 | | — | 0.5 | — | — | 75 | — |
| 21 | — | — | | — | 0.5 | — | — | 75 | — |
| 22 | — | — | — | 1.2 | — | — | — | ≧70 | — |
| 23 | — | — | — | 1.2 | — | — | — | ≧70 | — |
| 24 | — | — | — | 1.4 | — | — | — | ≧70 | — |
| 25 | — | — | — | 1.4 | — | — | — | ≧70 | — |
| 26 | — | — | | — | 0.3 | — | — | 75 | — |
| 27 | 7.5 | 2.5 | | — | 0.5 | — | — | 75 | — |
| 28 | — | — | | — | 0.5 | — | — | 75 | — |
| 29 | — | — | | — | 0.5 | — | — | 75 | — |
| 30 | 7.5 | 2.5 | | — | 0.5 | — | — | 75 | — |
| 31 | — | — | | — | 0.5 | — | 15 | 90 | — |
| 32 | 7.4 | 2.5 | — | 1.2 | — | — | — | — | 20 |
| 33 | 7.5 | 2.5 | | — | 0.3 | — | — | 90 | — |
| 34 | — | — | — | 1.2 | — | — | — | ≧70 | |
| 35 | — | — | — | 1.0 | — | — | — | — | 20 |
| 36 | — | — | — | 1.2 | — | — | — | ≧70 | |
| 37 | — | — | | — | 0.6 | — | 10 | 90 | — |
| 38 | 7.5 | 2.5 | | — | 0.5 | — | — | 100 | — |
| 39 | — | — | | — | 0.5 | — | 15 | 90 | — |
| 40 | — | — | | — | 0.5 | — | 15 | 90 | — |
| 41 | 9.8 | — | — | 1.2 | — | — | — | ≧70 | — |
| 42 | — | — | | — | 0.5 | — | 15 | 90 | — |
| 43 | — | — | | — | 0.5 | — | 15 | 90 | — |
| 44 | 10 | — | | 1.2 | — | — | — | — | 20 |
| 45 | 20 | — | | 1.2 | — | — | — | — | 20 |
| 46 | 30 | — | | 1.2 | — | — | — | — | 20 |
| 47 | 35 | — | | 1.2 | — | — | — | — | 20 |
| 48 | — | — | | 1.2 | — | — | 5 | 80 | — |
| 49 | — | — | | 1.2 | — | — | 10 | 80 | — |
| 50 | — | — | | 1.2 | — | — | 10 | 80 | — |

| | |
|---|---|
| Ex. | Example |
| ^{#} | The amounts given are in % by weight, based on the compounds used for |
| | the polymerization + Si-containing thickener |
| (EO)-silicone | Belsil ® 6031, ethoxylated dimethylsiloxane; |
| SI-UPA | Silicone-containing urethane-polyallyl |
| VP | Vinylpyrrolidone |
| VCap | N-Vinylcaprolactam |
| MAA | Methacrylic acid |
| AA | Acrylic acid |
| VI | Vinylimidazole |
| DMAPMAM | Dimethylaminopropylmethacrylamide |
| SMA | Stearyl methacrylate |
| MMA | Methyl methacrylate |
| C₁₈-PEG-MA | Polyethylene glycol methacrylate terminated with a C₁₈-fatty alcohol |
| BMA | n-Butyl methacrylate |
| EGDMA | Ethylene glycol dimethacrylate |
| PETAE | Pentaerythritol triallyl ether |
| PEG 9000 | Polyethylene glycol, M_{w} = 9 000 |
| Lutensol ® AT 25 | C₁₆—C₁₈-fatty alcohol ethoxylate with 25 EO units |
| Quat. | Quaternization |
| Neut. | Neutralization |
| TEA | Triethylamine |
| DN % | Degree of neutralization in % |

### EXAMPLE 83

### (Variant B): Polymerization of VI/MAS/VP/PETAE in the Presence of an Ethoxylated Dimethylsiloxane Using Two Free-Radical Initiators with Varying Decomposition Temperature

| | | |
|---|---|---|
| Initial charge: | 613 g | butyl acetate |
| | 2 g | Belsil ® DMC 6031 |
| | 1 g | Trigonox ® 101 (2,5-dimethyl- |
| | | 2,5-di(tert-butylperoxy)hexane) |
| Feed 1: | 94 g | vinylpyrrolidone |
| | 102 g | vinylimidazole |
| | 6.95 g | methacrylic acid |
| | 1.2 g | pentaerythritol triallyl ether |
| Feed 2: | 35 g | n-butyl acetate |
| | 0.2 g | tert-butyl peroctoate |
| Feed 3: | 175 g | n-butyl acetate |
| | 1.0 g | tert-butyl peroctoate |
| Feed 4: | 175 g | n-butyl acetate |
| | 1.0 g | tert-butyl peroctoate |

The initial charge was heated to 90° C. under a nitrogen atmosphere in an apparatus fitted with stirrer, reflux counter, internal thermometer and four feed devices. Feeds 1 and 2 were added over the course of 3 h and the mixture was stirred at 90° C. for a further 1.5 h. Feed 3 was added at 100° C. over the course of 1 h, and the reaction mixture was stirred at this temperature for 1 h. Then, feed 4 was added at 100° C. over the course of 1 h and the mixture was then stirred again for 2 h at 100° C. The temperature was increased to 125° C. and the mixture was further stirred at this temperature for a further 2 h. The resulting white suspension was then quaternized with 50 g of methyl chloride. The product was filtered, washed with acetone and dried under reduced pressure at 70° C.

All of the products of variant B as in Table 2 were produced analogously.

### EXAMPLE 73

### (Variant C): Polymerization of AA/DMAPMAM/PETAE in the Presence of an Ethoxylated Dimethylsiloxane Using Two Free-Radical Initiators with Varying Decomposition Temperature

| | | |
|---|---|---|
| Initial charge: | 800 g | ethyl acetate |
| | 1.6 g | Belsil ® DMC 6031 |
| | 1 g | tert-butyl peroctoate |
| Feed 1: | 125 g | acrylic acid |
| Feed 2: | 45 g | DMAPMAM |
| | 1.6 g | pentaerythritol triallyl ether |
| Feed 3: | 80 g | ethyl acetate |
| | 0.4 g | lauroyl peroxide |
| Feed 4: | 200 g | ethyl acetate |
| | 0.4 g | lauroyl peroxide |

The initial charge was heated to 75° C. under a nitrogen atmosphere and with stirring in an apparatus equipped with stirrer, reflux counter, internal thermometer and four feed devices. Feeds 1, 2 and 3 were added over the course of 3 h and the mixture was stirred for a further 2 h at 75° C. Feed 4 was added over 1 h at 80° C. and the mixture was then further stirred for 1 h. The temperature was increased to 100° C. and the mixture was stirred for a further 3 h at this temperature. The resulting white suspension was then quaternized with 40 g of methyl chloride. The product was filtered, washed with acetone and dried under reduced pressure at 70° C.

All of the products of variant C as in Table 2 were produced analogously.

### EXAMPLE 52

### (Variant D): Polymerization of AA/DMAPMAM/SMA/PETAE in the Presence of an Ethoxylated Dimethylsiloxane Using Two Free-Radical Initiators with Varying Decomposition Temperature

| | | |
|---|---|---|
| Initial charge: | 670 g | ethyl acetate/cyclohexane (65:35) |
| | 2 g | Belsil ® DMC 6031 |
| | 50 g | feed 1 |
| | 14 g | feed 2 |
| | 1.5 g | pentaerythritol triallyl ether |
| | 1.5 g | tert-butyl peroctoate |
| Feed 1: | 142.5 g | acrylic acid |
| | 3 g | stearyl methacrylate |
| | 3 g | dimethylaminopropylmethacrylamide |
| | 100 g | ethyl acetate/cyclohexane (65:35) |
| | 4.3 g | anhydrous K₂CO₃ |
| Feed 2: | 70 g | ethyl acetate/cyclohexane (65:35) |
| | 0.35 g | Trigonox ® EHP-C75 (75% strength) |
| Feed 3: | 70 g | ethyl acetate/cyclohexane (65:35) |
| | 1.0 g | Trigonox ® EHP-C75 (75% strength) |

The initial charge was heated to 50° C. under a nitrogen atmosphere and with stirring in an apparatus fitted with stirrer, reflux counter, internal thermometer and three feed devices. Feed 1 was added over the course of 1.5 h and feed 2 was added over the course of 2 h and the mixture was stirred at 60° C. for a further 2 h. Feed 3 was added over 1 h at 60° C. and the mixture was then stirred for a further 2 h at 70° C. The temperature was increased to 100° C. and the mixture was further stirred for 3 h at the temperature. The resulting white suspension was filtered, washed with acetone and dried at 70° C. under reduced pressure.

All of the products of variant D as in Table 2 were produced analogously.

TABLE 2

| | | | | | | | | | | | | | Pro- |
|---|---|---|---|---|---|---|---|---|---|---|---|---|---|
| | (EO) | | | | | | | | | | | | duction |
| Ex. | silicone^{#} | VP^{#} | MAA# | AA# | VI^{#} | DMAPMAM^{#} | SMA^{#} | n-BA^{#} | EMA^{#} | PLEX-O^{#} | EGDMA^{#} | PETAE^{#} | variant |
| 51 | 2 | — | 90 | — | 4 | — | 5 | — | — | — | 1.0 | — | C |
| 52 | 2 | — | — | 95 | — | 2 | 2 | — | — | — | — | 1.0 | D |
| 53 | 2 | — | 85 | — | 10 | — | 3.5 | — | — | — | 1.5 | — | C |
| 54 | 2 | — | — | 92 | — | 4 | 2.5 | — | — | — | — | 1.5 | D |
| 55 | 2 | — | — | 85 | — | 12 | 1.8 | — | — | — | — | 1.2 | C |
| 56 | 2 | — | 85 | — | 5 | 5 | 3.8 | — | — | — | 1.2 | — | C |
| 57 | 2 | — | 78 | — | 10 | 7 | 3.8 | — | — | — | 1.2 | — | B |
| 58 | 2 | — | — | 75 | — | 10 | — | — | 13.5 | — | — | 1.5 | C |
| 59 | 1 | — | 75 | — | — | 23.5 | — | — | — | — | 1.5 | — | C |
| 60 | 1 | — | 70 | — | — | 23.5 | — | — | — | 5 | 1.5 | — | C |
| 61 | 1 | — | 45 | — | — | 48.5 | — | — | — | 5 | 1.5 | — | C |
| 62 | 1 | — | 50 | — | 25 | 23.8 | — | — | — | — | 1.2 | — | C |
| 63 | 1 | — | 50 | — | 25 | 20 | 3.8 | — | — | — | 1.2 | — | C |
| 64 | 1 | — | 45 | — | — | 38.5 | — | 15 | — | — | 1.5 | — | C |
| 65 | 1 | — | 45 | — | — | 38.5 | — | 10 | — | 5 | 1.5 | — | C |
| 66 | 1 | — | 45 | — | — | 33.5 | — | 15 | — | 5 | 1.5 | — | C |
| 67 | 1 | — | 45 | — | — | 33.5 | — | 25 | — | — | 1.5 | — | C |
| 68 | 1 | — | 45 | — | — | 28.5 | — | 25 | — | — | 1.5 | — | C |
| 69 | 1 | — | 45 | — | — | 28 | — | 25 | — | — | 2 | — | C |
| 70 | 1 | — | 45 | — | — | 23.5 | — | 30 | — | — | 1.5 | — | C |
| 71 | 1 | — | 40 | — | — | 23.5 | — | 35 | — | — | 1.5 | — | C |
| 72 | 1 | — | 70 | — | — | 28.8 | — | — | — | — | 1.2 | — | C |
| 73 | 1 | — | — | 70 | — | 28.8 | — | — | — | — | — | 1.2 | C |
| 74 | 1 | 25 | 70 | — | — | 3.8 | — | — | — | — | 1.2 | — | B |
| 75 | 1 | 25 | — | 70 | — | 3.8 | — | — | — | — | 1.2 | — | B |
| 76 | 1 | 25 | 50 | — | — | 23.8 | — | — | — | — | 1.2 | — | C |
| 77 | 1 | 25 | — | 50 | — | 23.8 | — | — | — | — | — | 1.2 | C |
| 78 | 1 | 45 | 50 | — | — | 3.8 | — | — | — | — | 1.2 | — | C |
| 79 | 1 | 45 | — | 50 | — | 3.8 | — | — | — | — | — | 1.2 | C |
| 80 | 1 | 45 | 30 | — | — | 23.8 | — | — | — | — | 1.2 | — | C |
| 81 | 1 | 45 | — | 30 | — | 23.8 | — | — | — | — | 1.2 | — | C |
| 82 | 1 | 61 | 3.3 | — | 35 | — | — | — | — | — | — | 0.7 | B |
| 83 | 1 | 46 | 3.3 | — | 50 | — | — | — | — | — | — | 0.7 | B |
| 84 | 1 | 31 | 3.3 | — | 65 | — | — | — | — | — | — | 0.7 | B |
| 85 | 1 | 16 | 3.3 | — | 80 | — | — | — | — | — | — | 0.7 | B |
| 86 | 2 | — | 4.5 | — | 85 | — | — | — | 10 | — | — | 0.5 | B |
| 87 | 2 | — | 5.5 | — | 94 | — | — | — | — | — | — | 0.5 | B |
| 88 | 2 | — | 5.5 | — | 90 | — | 4 | — | 4 | — | — | 0.5 | B |
| 89 | 1 | — | 3.3 | — | 93 | — | 3 | — | 3 | — | — | 0.7 | B |
| 90 | 1 | — | 2.3 | — | 95 | — | 2 | — | 2 | — | — | 0.7 | B |

| | |
|---|---|
| Ex. | Example |
| ^{#} | The amounts given are in % by weight, based on the unsaturated compounds used for the polymerization, |
| | the parts by weigth of Si-containing compound are given separately |
| (EO) silicone | Belsil ® 6031, ethoxylated dimethylsiloxane |
| VP | vinylpyrrolidone |
| MAA | methacrylic acid |
| AA | acrylic acid |
| VI | vinylimidazole |
| DMAPMAM | dimethylaminopropylmethacrylamide |
| SMA | stearyl methacrylate |
| EGDMA | ethylene glycol dimethacrylate |
| PETAE | pentaerythritol triallyl ether |
| EMA | ethyl methacrylate |
| n-BA | n-butyl acrylate |
| PLEX-O | Plex ® 6877-0 = methacrylic acid ester of a C₁₆—C₁₈-fatty alcohol alkoxylated with 25 mol of ethylene oxide, |
| | as 25% strength solution in methyl methacrylate |

1. A copolymer A) containing silicone groups, obtainable by free-radical copolymerization of
a) at least one compound with a free-radically polymerizable, α,β-ethylenically unsaturated double bond and at least one ionogenic and/or ionic group per molecule,
b) at least one free-radically polymerizable crosslinking compound which comprises at least two α,β-ethylenically unsaturated double bonds per molecule,
in the presence of at least one silicone compound c) which comprises a polyether group and/or a free-radically polymerizable olefinically unsaturated double bond.

2. The copolymer A) according to claim 1, which is obtainable by free-radical copolymerization in accordance with the method of precipitation polymerization.

3. The copolymer according to claim 1, where the component a) comprises at least one compound a1) with at least one anionogenic and/or anionic group which is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof.

4. The copolymer according to claim 1, where the component a) comprises at least one compound a2) with at least one cationogenic and/or cationic group which is chosen from esters of α,β-ethylenically unsaturated mono- and dicarboxylic acids with amino alcohols, which may be mono- or dialkylated on the amine nitrogen, amides of α,β-ethylenically unsaturated mono- and dicarboxylic acids with diamines which have at least one primary or secondary amino group, N,N-diallylamine, N,N-diallyl-N-alkylamines and derivatives thereof, vinyl- and allyl-substituted nitrogen heterocycles, vinyl- and allyl-substituted heteroaromatic compounds and mixtures thereof.

5. The copolymer according to claim 1, where the component a) comprises at least one compound with at least one anionogenic and/or anionic group a1) and at least one compound with at least one cationogenic and/or cationic group a2).

6. The copolymer according to claim 1, which additionally comprises at least one further monomer d) in copolymerized form which is chosen from α,β-ethylenically unsaturated compounds containing amide groups and of the general formula I 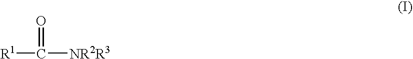
where
one of the radicals R¹ to R³ is a group of the formula CH₂═CR⁴— where R⁴ is H or C₁-C₄-alkyl and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8.

7. The copolymer according to claim 1, which additionally comprises at least one compound e) in copolymerized form which is chosen from compounds of the general formulae III a), III b), III c), III d) and III e) 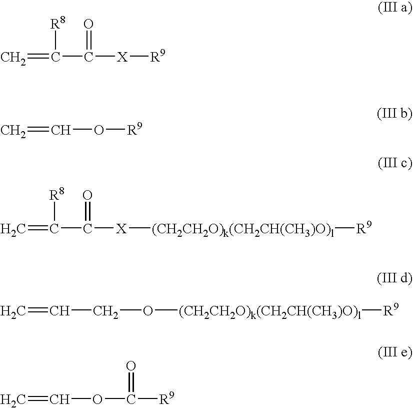
in which
the order of the alkylene oxide units is arbitrary,
k and l, independently of one another, are an integer from 0 to 1000, where the sum of k and l is at least 5,
R⁸ is hydrogen or C₁-C₄-alkyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

8. The copolymer according to claim 1, which additionally comprises at least one compound f) in copolymerized form which is chosen from compounds of the general formulae III a*), III b*), III c*), III d*) and III e*) 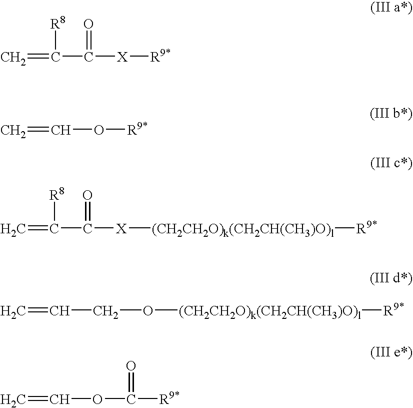
in which
the order of the alkylene oxide units is arbitrary,
k and l, independently of one another, are an integer from 0 to 1000, where the sum of k and l is at least 5,
R⁸ is hydrogen or C₁-C₄-alkyl,
R⁹* is hydrogen, C₁-C₈-alkyl or C₃-C₈-alkenyl, and
X is O or a group of the formula NR¹⁰, in which R¹⁰ is H, alkyl, alkenyl, cycloalkyl, heterocycloalkyl, aryl or hetaryl.

9. The copolymer according to claim 1, which additionally comprises at least one compound g) in copolymerized form which is chosen from esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols having a primary or secondary amino group, esters of allyl alcohol with C₁-C₇-monocarboxylic acids, polyether acrylates different from III c) and III c*), vinylaromatics, vinyl halides, vinylidene halides, C₁-C₈-monoolefins, nonaromatic hydrocarbons with at least two conjugated double bonds and mixtures thereof.

10. The copolymer A) according to claim 1, which comprises, in copolymerized form,
at least 2% by weight of at least one compound a1) with a free-radically polymerizable, α,β-ethylenically unsaturated double bond and at least one anionogenic and/or anionic group per molecule,
0.05 to 5% by weight of at least one crosslinker b), preferably ethylene glycol di(meth)acrylate and/or pentaerythritol triallyl ether,
0.05 to 30% by weight of at least one silicone compound c).

11. The copolymer A) according to claim 1, which comprises, in copolymerized form,
at least 2% by weight of at least one compound a1) with a free-radically polymerizable, α,β-ethylenically unsaturated double bond and at least one anionogenic and/or anionic group per molecule,
0.05 to 5% by weight of at least one crosslinker b),
0.05 to 30% by weight of at least one silicone compound c),
20 to 95% by weight of vinylpyrrolidone d),
0.1 to 20% by weight of at least one compound e),
5 to 40% by weight of at least one monomer f).

12. The copolymer A) according to claim 1, obtainable by free-radical copolymerization of
at least 2% by weight, based on the total weight of the monomers used for the polymerization, of at least one monomer pair of at least one N-vinylimidazole compound a2) and acrylic acid and/or methacrylic acid a1),
at least 1% by weight, based on the total weight of the monomers used for the polymerization, of at least one additional monomer a1) with an anionogenic or anionic group or at least one additional monomer a2) with a cationogenic or cationic group,
0.05 to 5% by weight, based on the total weight of the monomers used for the polymerization, of at least one free-radically polymerizable crosslinking compound b),
0.05 to 30% by weight, of at least one silicone compound c),
0 to 95% by weight of at least one monomer d) which contains amide groups,
0 to 40% by weight, based on the total weight of the monomers used for the polymerization, of at least one hydrophobic monomer e),
0 to 40% by weight, based on the total weight of the monomers used for the polymerization, of at least one monomer f).

13. The copolymer A) according to claim 1, obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 70% by weight of at least one additional monomer with a cationogenic or cationic group a2),
0.1 to 2% by weight of at least one crosslinker b),
0.05 to 30% by weight, of at least one silicone compound c),
0 to 95% by weight, preferably 20 to 95% by weight, of vinylpyrrolidone and/or vinylcaprolactam d),
0 to 20% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer f) which is preferably chosen from C₁-C₆-(meth)acrylates, in particular methyl (meth)acrylate, ethyl (meth)acrylate, n-butyl (meth)acrylate and mixtures thereof.

14. A process for the preparation of a copolymer A) containing silicone groups as defined in claim 1, by free-radical copolymerization in accordance with the method of precipitation polymerization.

15. The process according to claim 14, where the polymerization takes place in a largely anhydrous, aprotic solvent or solvent mixture.

16. The process according to claim 14, in which, for the copolymerization, at least two initiators are used whose decomposition temperatures are different from one another by at least 10° C.

17. The process according to claim 16, where the copolymerization takes place until completion of the precipitation of the copolymer at a temperature greater than or equal to the lower decomposition temperature and less than the higher decomposition temperature and, after the precipitation, a further reaction takes place at a temperature greater than or equal to the higher decomposition temperature.

18. The process according to claim 14, comprising a first polymerization phase at a first polymerization temperature and a second polymerization phase at a second polymerization temperature above the first polymerization temperature, where, for the polymerization, at least two initiators are used whose half-lives at the first polymerization temperature differ in such a way that at least one of these initiators decomposes into free radicals during the first polymerization phase and at least one of these initiators essentially does not decompose into free radicals during the first polymerization phase and decomposes into free radicals during the second polymerization phase.

19. A cosmetic or pharmaceutical composition comprising
A) at least one copolymer containing silicone groups as defined in claim 1,
B) at least one cosmetically or pharmaceutically acceptable active substance or effect substance, and
C) if appropriate at least one further cosmetically or pharmaceutically acceptable auxiliary different from B).

20. The copolymer according to claim 7, wherein R⁸ is methyl.
